SEQUENCE LISTING (66 sequences)

SEQ ID NO: 1 - protein, 100 aa, Artificial
  Engineered C1
PTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 2 - DNA, 300 bp, Artificial
  Engineered C1
cccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 3 - protein, 100 aa, Artificial; identical to SEQ ID NO: 1
  Engineered C2

SEQ ID NO: 4 - DNA, 300 bp, Artificial
  Engineered C2
ccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 5 - protein, 100 aa, Artificial
  Engineered C3
PSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 6 - DNA, 300 bp, Artificial
  Engineered C3
ccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 7 - protein, 100 aa, Artificial
  Engineered C6
PSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 8 - DNA, 300 bp, Artificial
  Engineered C6
ccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 9 - protein, 100 aa, Artificial
  Engineered C7
PSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 10 - DNA, 300 bp, Artificial
  Engineered C7
ccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 11 - protein, 96 aa, Artificial
  Engineered IgG1 CH1
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 12 - DNA, 289 bp, Artificial
  Engineered IgG1 CH1
accaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttg

SEQ ID NO: 13 - protein, 96 aa, Artificial
  Engineered IgG2 CH1
TKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 14 - DNA, 288 bp, Artificial
  Engineered IgG2 CH1
accaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagtt

SEQ ID NO: 15 - protein, 96 aa, Artificial
  Engineered IgG3 CH1
TKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 16 - DNA, 288 bp, Artificial
  Engineered IgG3 CH1
accaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagtt

SEQ ID NO: 17 - protein, 96 aa, Artificial
  Engineered IgG4 CH1
TKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 18 - DNA, 288 bp, Artificial
  Engineered IgG4 CH1
accaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagtt

SEQ ID NO: 19 - protein, 4 aa, Artificial
  Conjunction domain 1
SSAS

SEQ ID NO: 20 - DNA, 12 bp, Artificial
  Conjunction domain 1
tcgtcggcttca

SEQ ID NO: 21 - protein, 5 aa, Artificial
  Conjunction domain 2
SSASS

SEQ ID NO: 22 - DNA, 15 bp, Artificial
  Conjunction domain 2
tcgtcggcttcatcg

SEQ ID NO: 23 - protein, 6 aa, Artificial
  Conjunction domain 3
SSASSS

SEQ ID NO: 24 - DNA, 18 bp, Artificial
  Conjunction domain 3
tcgtcggcttcatcgtca

SEQ ID NO: 25 - protein, 7 aa, Artificial
  Conjunction domain 4
SSASKAA

SEQ ID NO: 26 - DNA, 21 bp, Artificial
  Conjunction domain 4
agttcggcctcaaaggctgcc

SEQ ID NO: 27 - protein, 8 aa, Artificial
  Conjunction domain 5
SSASSKAA

SEQ ID NO: 28 - DNA, 24 bp, Artificial
  Conjunction domain 5
tcgtcggcttcatcgaaggctgcc

SEQ ID NO: 29 - protein, 9 aa, Artificial
  Conjunction domain 6
SSASSSKAA

SEQ ID NO: 30 - DNA, 27 bp, Artificial
  Conjunction domain 6
tcgtcggcttcatcgtcaaaggctgcc

SEQ ID NO: 31 - protein, 8 aa, Artificial
  Conjunction domain 7
EDLNKVFP

SEQ ID NO: 32 - DNA, 24 bp, Artificial
  Conjunction domain 7
gaggacctgaacaaggtgttccca

SEQ ID NO: 33 - protein, 8 aa, Artificial
  Conjunction domain 9
EDLSNVSP

SEQ ID NO: 34 - DNA, 24 bp, Artificial
  Conjunction domain 9
gaggacctgtccaatgtcagtccc

SEQ ID NO: 35 - protein, 8 aa, Artificial
  Conjunction domain 8
EDLKNVFP

SEQ ID NO: 36 - DNA, 24 bp, Artificial
  Conjunction domain 8
gaggacctgaaaaacgtgttccca

SEQ ID NO: 37 - protein, 117 aa, Artificial
  CAb1-NY-ESO-1_V
AQSVAQPEDQVNVAEGNPLTVKCTYSVSGNPYLFWYVQYPNRGLQFLLKYLGDSALVKGSYGFEAEFNKSQTSFHLKKPSALVSDSALYFCAVRDIRSGAGSYQLTFGKGTKLSVIP

SEQ ID NO: 38 - DNA, 351 bp, Artificial
  CAb1-NY-ESO-1_V
gcccagtccgtggctcagcccgaggaccaagtgaacgtggccgagggcaaccctctgaccgtgaagtgcacctattccgtgagcggcaacccctatctgttttggtacgtgcagtaccccaacagaggactgcagtttctgctgaagtatctgggagacagcgctctggtgaagggaagctacggcttcgaagccgagttcaacaagagccagacctccttccatctgaagaagcctagcgctctggtgagcgactccgctctgtacttctgcgccgtcagagacatcagaagcggcgccggaagctaccagctgaccttcggcaagggcaccaagctgagcgtgatccct

SEQ ID NO: 39 - protein, 115 aa, Artificial
  CAb1-NY-ESO-1_V
SAVISQKPSRDIKQRGTSLTIQCQVDKRLALMFWYRQQPGQSPTLIATAWTGGEATYESGFVIDKFPISRPNLTFSTLTVSNMSPEDSSIYLCSVGGSGAADTQYFGPGTRLTVL

SEQ ID NO: 40 - DNA, 345 bp, Artificial
  CAb1-NY-ESO-1_V
agcgccgtgatcagccagaagcctagcagagacatcaaacagaggggcacatctctgaccatccagtgccaagtggacaagagactcgctctgatgttctggtatagacagcagcccggacagtcccccacactgatcgccaccgcttggaccggcggagaagccacctacgagtccggcttcgtgatcgacaagttccccatctctagacccaatctgaccttttccacactgaccgtgtccaacatgagccccgaggactccagcatttatctgtgtagcgtgggaggcagcggagctgccgatacccagtacttcggccccggaaccagactgaccgtgctg

SEQ ID NO: 41 - protein, 109 aa, Artificial
  CAb2-GP100_V
AQQGEEDPQALSIQEGENATMNCSYKTSINNLQWYRQNSGRGLVHLILIRSNEREKHSGRLRVTLDTSKKSSSLLITASRAADTASYFCATDGSTPMQFGKGTRLSVIA

SEQ ID NO: 42 - DNA, 327 bp, Artificial
  CAb2-GP100_V
gctcagcaaggcgaagaggatccccaagctctgagcattcaagagggcgagaacgccaccatgaactgctcctacaagaccagcatcaacaacctccagtggtatagacagaacagcggcagaggactggtgcatctgattctgattagaagcaacgagagagagaagcactccggaaggctgagggtgacactggatacaagcaagaagagcagctctctgctgatcaccgcttccagagccgctgacaccgccagctacttctgcgccaccgacggcagcacccctatgcagttcggcaagggcacaagactcagcgtgatcgcc

SEQ ID NO: 43 - protein, 112 aa, Artificial
  CAb2-GP100_V
DGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVT

SEQ ID NO: 44 - DNA, 336 bp, Artificial
  CAb2-GP100_V
gacggcggcatcacccagtcccccaagtatctgtttagaaaggagggccagaatgtgacactgagctgcgagcagaatctgaaccacgacgccatgtactggtacagacaagaccccggccaaggactgaggctgatctattacagctgggcacaaggagacttccagaagggcgacatcgccgagggatacagcgtgtctagagagaagaaggagagctttcctctgaccgtgaccagcgcccagaagaatcccaccgccttctatctgtgtgccagcagctggggagctccctacgagcagtatttcggacccggcacaagactgaccgtgaca

SEQ ID NO: 45 - protein, 113 aa, Artificial
  CAb3-NY-ESO-1_V
QEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHP

SEQ ID NO: 46 - DNA, 339 bp, Artificial
  CAb3-NY-ESO-1_V
caagaagtgacacagatccctgccgctctgtctgtgcctgagggcgaaaacctggtgctgaactgcagcttcaccgacagcgccatctacaacctgcagtggttcagacaggaccccggcaagggactgacaagcctgctgctgattaccccttggcagagagagcagaccagcggcagactgaatgccagcctggataagtcctccggcagaagcaccctgtatatcgccgcttctcagcctggcgatagcgccacatatctgtgtgccgtcagacccctgctggacggcacatatatccccacctttggcagaggcaccagcctgatcgtgcaccct

SEQ ID NO: 47 - protein, 111 aa, Artificial
  CAb3-NY-ESO-1_V
GVTQTPKFQVLKTGQSMTLQCAQDMNHEYMSWYRQDPGMGLRLIHYSVAIQTTDRGEVPNGYNVSRSTIEDFPLRLLSAAPSQTSVYFCASSYLGNTGELFFGEGSRLTVL

SEQ ID NO: 48 - DNA, 333 bp, Artificial
  CAb3-NY-ESO-1_V
ggagttacacagacccctaagttccaggtgctgaaaaccggccagagcatgaccctgcagtgcgcccaggatatgaaccacgagtacatgagctggtacaggcaggatccaggcatgggcctgagactgatccactactctgtggccatccagaccaccgacagaggcgaagtgcccaacggctacaacgtgtccagatccaccatcgaggacttcccactgagactgctgtctgctgcccctagccagacctccgtgtacttttgtgccagcagctacctgggcaacaccggcgagctgttttttggcgagggctccagactgaccgtgctg

SEQ ID NO: 49 - protein, 253 aa, Artificial
  Anti-CD3-scFv
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 50 - DNA, 759 bp, Artificial
  Anti-CD3-scFv
gccatccagatgacgcaaagtccatcaagtctgagcgccagcgtgggcgacagagtgaccatcacctgcagagccagccaggacatcagaaattacctgaattggtaccagcagaagcctggcaaggctccaaagctcctcatatattatacatcgagattagaatctggtgttccaagcagattcagcggcagcggcagcggcaccgactacaccctgaccatcagcagcctgcagcctgaggacttcgccacctactactgccagcagggcaataccctgccttggacatttggacagggtaccaaggtggaaattaaaggcggcggcggaagcggaggcggagggtcgggtggcggaggttcaggtggaggagggtctggtggaggctcagaggtacaacttgtggagtcaggcggtggactagtccaaccaggaggatctttacgcttatcttgtgccgccagcggctacagcttcaccggctacaccatgaattgggtgagacaggctcccggtaagggcctggagtgggtggccctgatcaatccttacaagggcgtgagcacctacaatcagaagttcaaggacagattcaccatcagcgtggacaagagcaagaataccgcctacctgcagatgaatagcctgagagccgaggacaccgccgtgtactactgcgccagaagcggctactacggcgacagcgactggtactttgatgtttgggggcaaggtacacttgtcactgtaagctcc

SEQ ID NO: 51 - protein, 203 aa, Artificial
  CAb1-NY-ESO-1_FL
AQSVAQPEDQVNVAEGNPLTVKCTYSVSGNPYLFWYVQYPNRGLQFLLKYLGDSALVKGSYGFEAEFNKSQTSFHLKKPSALVSDSALYFCAVRDIRSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 52 - DNA, 609 bp, Artificial
  CAb1-NY-ESO-1_FL
gcccagtccgtggctcagcccgaggaccaagtgaacgtggccgagggcaaccctctgaccgtgaagtgcacctattccgtgagcggcaacccctatctgttttggtacgtgcagtaccccaacagaggactgcagtttctgctgaagtatctgggagacagcgctctggtgaagggaagctacggcttcgaagccgagttcaacaagagccagacctccttccatctgaagaagcctagcgctctggtgagcgactccgctctgtacttctgcgccgtcagagacatcagaagcggcgccggaagctaccagctgaccttcggcaagggcaccaagctgagcgtgatccctaacatccagaaccccgatcccgccgtgtaccagctgagggacagcaagtccagcgacaagtccgtgtgtctgttcaccgacttcgactcccagaccaacgtgtcccagagcaaggatagcgacgtgtacatcaccgacaagtgcgtcctcgacatgaggtccatggacttcaagagcaacagcgccgtggcttggagcaacaagagcgacttcgcttgcgccaacgccttcaacaacagcatcatccccgaggacacc

SEQ ID NO: 53 - protein, 245 aa, Artificial
  CAb1-NY-ESO-1_FL
SAVISQKPSRDIKQRGTSLTIQCQVDKRLALMFWYRQQPGQSPTLIATAWTGGEATYESGFVIDKFPISRPNLTFSTLTVSNMSPEDSSIYLCSVGGSGAADTQYFGPGTRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 54 - DNA, 735 bp, Artificial
  CAb1-NY-ESO-1_FL
agcgccgtgatcagccagaagcctagcagagacatcaaacagaggggcacatctctgaccatccagtgccaagtggacaagagactcgctctgatgttctggtatagacagcagcccggacagtcccccacactgatcgccaccgcttggaccggcggagaagccacctacgagtccggcttcgtgatcgacaagttccccatctctagacccaatctgaccttttccacactgaccgtgtccaacatgagccccgaggactccagcatttatctgtgtagcgtgggaggcagcggagctgccgatacccagtacttcggccccggaaccagactgaccgtgctggaggatctgaagaacgtgtttccccccgaggtggccgtgtttgagcccagcgaggccgagattagccacacccagaaggccacactggtgtgtctggccaccggcttttaccccgaccacgtggaactgagctggtgggtgaacggcaaggaggtgcactccggcgtgtgtaccgatccccagcctctgaaggagcagcccgccctcaacgatagcagatacgctctgtcctccagactgagagtgagcgccacattctggcaagaccccagaaaccactttagatgccaagtgcagttctacggactgagcgaaaacgacgagtggacacaagatagagccaagcccgtgacccagatcgtgagcgccgaggcttggggcagagccgat

SEQ ID NO: 55 - protein, 195 aa, Artificial
  CAb2-GP100_FL
AQQGEEDPQALSIQEGENATMNCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 56 - DNA, 585 bp, Artificial
  CAb2-GP100_FL
gctcagcaaggcgaagaggatccccaagctctgagcattcaagagggcgagaacgccaccatgaactgctcctacaagaccagcatcaacaacctccagtggtatagacagaacagcggcagaggactggtgcatctgattctgattagaagcaacgagagagagaagcactccggaaggctgagggtgacactggatacaagcaagaagagcagctctctgctgatcaccgcttccagagccgctgacaccgccagctacttctgcgccaccgacggcagcacccctatgcagttcggcaagggcacaagactcagcgtgatcgccaacatccagaagcccgaccccgccgtgtaccagctgagagactccaagagcagcgacaagagcgtgtgtctgttcaccgacttcgactcccagaccaacgtgagccagtccaaggacagcgacgtgtacatcaccgacaagtgcgtgctggacatgaggagcatggacttcaagtccaacagcgccgtggcttggtccaacaaatccgatttcgcttgcgccaatgccttcaacaactccatcatccccgaggacaca

SEQ ID NO: 57 - protein, 242 aa, Artificial
  CAb2-GP100_FL
DGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 58 - DNA, 726 bp, Artificial
  CAb2-GP100_FL
gacggcggcatcacccagtcccccaagtatctgtttagaaaggagggccagaatgtgacactgagctgcgagcagaatctgaaccacgacgccatgtactggtacagacaagaccccggccaaggactgaggctgatctattacagctgggcacaaggagacttccagaagggcgacatcgccgagggatacagcgtgtctagagagaagaaggagagctttcctctgaccgtgaccagcgcccagaagaatcccaccgccttctatctgtgtgccagcagctggggagctccctacgagcagtatttcggacccggcacaagactgaccgtgacagaggatctgaagaacgtcttccctcccgaggtggctgtgttcgagccctccgaggccgagatctcccacacccagaaggccaccctcgtgtgtctggctaccggcttctaccccgaccacgtggagctgagctggtgggtgaacggcaaagaggtgcatagcggcgtgtgtaccgacccccagcctctgaaagagcaacccgctctgaacgactccagatacgctctgtcctccagactgagggtctccgccacattttggcaagaccctagaaaccactttagatgtcaagtgcagttctacggactgagcgagaatgatgagtggacacaagacagagccaagcccgtgacacagattgtcagcgccgaggcttggggaagagctgat

SEQ ID NO: 59 - protein, 113 aa, Artificial; identical to SEQ ID NO: 45
  CAb3-NY-ESO-1_V

SEQ ID NO: 60 - DNA, 339 bp, Artificial; identical to SEQ ID NO: 46
  CAb3-NY-ESO-1_V

SEQ ID NO: 61 - protein, 111 aa, Artificial; identical to SEQ ID NO: 47
  CAb3-NY-ESO-1_V

SEQ ID NO: 62 - DNA, 333 bp, Artificial; identical to SEQ ID NO: 48
  CAb3-NY-ESO-1_V

SEQ ID NO: 63 - protein, 86 aa, Artificial
  C-alpha domain (protein)
NIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 64 - DNA, 258 bp, Artificial
  C-alpha domain (nucleic acid)
aacatccagaagcccgaccccgccgtgtaccagctgagagactccaagagcagcgacaagagcgtgtgtctgttcaccgacttcgactcccagaccaacgtgagccagtccaaggacagcgacgtgtacatcaccgacaagtgcgtgctggacatgaggagcatggacttcaagtccaacagcgccgtggcttggtccaacaaatccgatttcgcttgcgccaatgccttcaacaactccatcatccccgaggacaca

SEQ ID NO: 65 - protein, 130 aa, Artificial
  C-beta domain (protein)
EDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 66 - DNA, 390 bp, Artificial
  C-beta domain nucleic acid)
gaggatctgaagaacgtcttccctcccgaggtggctgtgttcgagccctccgaggccgagatctcccacacccagaaggccaccctcgtgtgtctggctaccggcttctaccccgaccacgtggagctgagctggtgggtgaacggcaaagaggtgcatagcggcgtgtgtaccgacccccagcctctgaaagagcaacccgctctgaacgactccagatacgctctgtcctccagactgagggtctccgccacattttggcaagaccctagaaaccactttagatgtcaagtgcagttctacggactgagcgagaatgatgagtggacacaagacagagccaagcccgtgacacagattgtcagcgccgaggcttggggaagagctgat

(19) United States
(12) Patent Application Publication
(10) Pub. No.: US 2023/0391846 A1
(43) Pub. Date: Dec. 7, 2023

(54) MODIFIED SOLUBLE T CELL RECEPTOR

(71) Applicant: WUXI BIOLOGICS IRELAND LIMITED, DUNDALK, CO LOUTH (IE)

(72) Inventors: Hanxiao YING, Shanghai (CN); Man XU, Shanghai (CN); Liying ZHANG, Shanghai (CN); Hanhan DU, Shanghai (CN); Juan LIU, Shanghai (CN); Jijie GU, Shanghai (CN); Gennady GOLOLOBOV, Shanghai (CN); Zhisheng CHEN, Shanghai (CN)

(21) Appl. No.: 18/033,789

(22) PCT Filed: Oct. 29, 2021
(86) PCT No.: PCT/CN2021/127290

§ 371 Date: Apr. 25, 2023

(30) Foreign Application Priority Data
Oct. 29, 2020 (CN) 202011180613.X

(51) Int. Cl. C07K 14/725; C07K 16/46; C12N 15/63; A61P 35/00

(52) U.S. Cl. CPC C07K 14/7051; C07K 16/468; C12N 15/63; A61P 35/00; C07K 2317/622; C07K 2317/31; C07K 2319/30

(57) ABSTRACT

The present invention provides an engineered chimeric soluble T cell receptor (ETCR), which comprises (i) all or part of TCR α chain, fused to all or part of the antibody constant domain, and (ii) all or part of TCR β chain, fused to all or part of the antibody constant domain. (i) and (ii) each comprise a designed linker, a designed binding interface between TCR and antibody domain and one or more mutagenesis in TCR domain to stabilize the ETCR thereof. Characterized in that the ETCR recognizes specific peptide-MHC (pMHC) complex and exhibits biological function.

### TECHNICAL FIELD

The present invention relates generally to engineered chimeric soluble T cell receptor and compositions thereof, and therapy in the treatment of disease.

### BACKGROUND OF THE INVENTION

T lymphocytes play a central role in adaptive immunity via respond to a wide variety of foreign antigens that are presented as peptides in the contest of major histocompatibility molecules (MHC). Specific recognition of peptide-MHC (pMHC) complexes is accomplished by a membrane-bound multicomponent, cell surface glycoprotein termed the T cell receptor (TCR). The native TCR is a heterodimeric cell surface protein of the immunoglobulin superfamily which is associated with invariant proteins of the CD3 complex involved in mediating signal transduction. TCRs exist in αβ and γδ forms, which are structurally similar but have quite distinct anatomical locations and probably functions. Particularly, αβ-TCR appears on over 95% of all T lymphocytes and assembled by almost unlimited repertoire diversities, providing pivotal protection for humans from both exogenous and endogenous diseases.

Antibodies and TCRs are the only two types of molecules which recognize antigens in a specific manner and TCR is the only receptor for particular peptide antigens presented in MHC, where the alien peptide often being the only sign of an abnormality within a cell. As with antibodies, there has also emerged an interest in developing soluble, antigen-specific TCR and its derivatives as drug candidates for expanding the therapeutics target to intracellular epitopes. Also, the specific TCR:pMHC interactions can be utilized as powerful diagnostic tool to detect infections, disease markers and specific cells with correspond pMHC complex expressed. However, unlike antibodies, TCRs are generally very unstable when expressed as soluble molecules, and problems such as low expression yields, aggregation and misfolding are often encountered. Possible explanations may include extensive glycosylation, instable constant domain and inefficient chain-pairing.

A number of papers described the production TCR heterodimers utilizing the native disulfide bridge in hinge region which connects the respective subunits (Garboczi et al., (1996), Nature 384(6605): 134-141; Garboczi et al., (1996), PNAS USA 91: 11408-11412; Davodeau et al, (1993), J. Biol Chem. 268(21): 15455-15460; Golden et al, (1997), J. Imm. Meth. 206: 163-169). However, although such TCRs can be recognized by TCR-specific antibodies, none were shown to recognize its native ligand, indicated a misfolded complementarity-determining regions (CDRs). Recently, in WO2004/074322, a soluble TCR is described which is correctly folded so that it is capable of recognizing its native ligand and stable over a period of time, and can be produced in reasonable quantities. This TCR comprises a TCR α chain extracellular domain dimerized to a TCR β chain extracellular domain which linked by an artificial disulfide bond between constant domain residues Cα S48-C β T57. Based on such soluble TCR format, a first-in-class bispecific TCR drug, Tebentafusp was developed and showed benefit to patients with metastatic melanoma. Similarly, in US2018/021682, another several artificial disulfide bonds between constant domain residues (Cα R53, P89, Y10 and Cβ S54, A19, and E20) and constant domain/viable domain residues (Vα 46, 47 (IMGT numbering) and Cβ 60, 61) were also described. Very recently, Karen et al. described a computational aided design of soluble TCR. Using both Rosetta calculation and experimental screening, they identified seven mutations in Cα and C α, which significantly improved the full-length TCR assembly and expression (Karen, et al., (2020), Nat. Comm., 11: 2330). Particularly, these soluble TCR designed based on modification of native TCR with either artificial disulfide bonds or mutagenesis are normally highly glycosylated especially in the constant domain, may potentially lead to uncertain performance as drug candidate. To avoid such shortcoming, in some cases, these soluble TCRs are produced in E co/i and assembled by protein re-folding process, resulted a relatively complicated manufacturing procedure.

The high degree of sequence identity between variable (V) and constant (C) domain of TCR and antibodies (30% to 70%) suggested that TCR are folded into β-sheet sandwich structures that would pair in a manner similar to the heavy (H) and light (L) chains of antibody Fab fragment. Considering the similar overall architecture and heterodimer association of TCR and antibody Fab, attempts have been made to generate the TCR-antibody chimeric proteins as an alternative way for obtaining soluble TCR. Previous described chimeric TCR format mainly includes a) directly infusion of all or part of TCR to fragment crystallizable (Fc) domain to make immunoglobulin like assembles and b) infusion of TCR V domain to antibody fab C domain with or without extra stabilizing domain (e.g. Fc region, leucine zipper) to make fab like assembles (Jack, et al, (1994), Proc. Natl. Acad. Sci USA 91: 12654-12658, Mark, et al, (1987), Proc. Natl Acad. Sci USA 84: 2936-2940, Greg, et al, (1988) J. Biol Chem. 264(13): 7310-7316, Bernard, et al, (1991), Proc. Natl Acad. Sci USA 88: 8077-8081, Jonathan, et al, (1997) J. Exp. Med 186(8): 1333-1345, Jonathan, et al., (1999) Cell Immunol 192: 175-184, AU729,406, U.S. Pat. No. 6,911,204). However, although correct function of these chimeric proteins was noted in few cases, extremely low expression level (30 ng/ml to 1 μg/ml) hindered its further applications as therapeutic protein. Indeed, many differences were revealed by a carefully inspection of the TCR and antibody structure, providing explanations for the unsatisfied result of previous simple infusion of TCR-antibody chimerics. The TCR is wider across the middle than an Fab (˜56 Å vs ˜46 Å) because of the protrusion of a loop in the Cβ domain that appears to be a general feature of all β chains. The TCR is also more asymmetric and squat than an Fab because of the more parallel crossing angle of the β-sheets into the Cα/Cβ interface, and the roughly 5 Å shift off-center in the position of the pseudo-2-fold relating C α/Cβ. This asymmetry is accentuated by the smaller size of the Cα domain as compared with the Cβ. Thus, instead of simple infusion of wildtype TCR and antibody, a comprehensive design based on the structure features is required for enhancing the compatibility thereby generating the stable and function chimerics.

Given the importance of soluble TCRs, it would be desirable to provide an alternative way of producing such molecules with native function and great developability. In the present invention, TCR (ETCR) and TCR derivatives (Bispecific ETCR) were produced stably, soluble and functionally in eukaryotic expression systems. Besides, strong in vitro anti-tumor activity was observed using thereof bispecific TCRs.

### BRIEF SUMMARY OF THE INVENTION

In one aspect, the present disclosure provides a polypeptide complex comprising a first polypeptide comprising, from N-terminus to C-terminus, a first TCR α chain variable domain of a first TCR operably linked to the first antibody constant domain (C1), and a second polypeptide comprising, from N-terminus to C-terminus, a first TCR β chain variable domain of a first TCR operably linked to the second antibody constant domain (C2), wherein C1 and C2 are capable of forming a dimer via its native inter-chain bonds and interactions. In certain embodiments, the first TCR has a first antigenic specificity.

In certain embodiments, the C1 and C2 comprises antibody heavy chain (CH1 domain) selecting from the group consisting of IgG1 (IMGT accession number: J00228, Z17370, AL122127, MG920252, MG92025, MG920246, MG920247, MG920248, MG920249, MG920250, MG920251, MG920253), IgG2 (IMGT accession number: J00230, AJ250170, AF449616, AF449618, AF928742, MH025828, MH025829, MH025830, MH025832, MH025833, MH025834, MH025835, MH025836), IgG3 (IMGT accession number: X03604, K01313, X16110, X99549, AJ390236, AJ390237, AJ390238, AJ390241, AJ390242, AL122127, AJ390247, AJ390252, AJ390254, AJ390260, AJ390262, AJ390272, AJ390276, MG920256, MG920255, MG920254, MH025837, MG920257, MG920258, MG920259, MG920260, MG786813, MG920261) IgG4 (IMGT accession number: K01316, AL928742), IgM (IMGT accession number: X14940, K01307, X57331, AC254827), IgA1 (IMGT accession number: J00220, IMGT000035), IgA2 (IMGT accession number: J00221, M60192, S71043), IgD (IMGT accession number: K02875, X57331), and IgE (IMGT accession number: J00222, L00022, IMGT000025, AL928742) or light chain constant domain (CA domain or CK domain) selecting from the group consisting of CA1 (IMGT accession number: J00252, X51755), CA2 (IMGT accession number: J00253, X06875, AJ491317), CA3 (IMGT accession number: J00254, K01326, X06876, D87017), CA6 (IMGT accession number: J03011), CA7 (IMGT accession number: X51755, M61771, X51755, M61771, KM455557), CK1 (IMGT accession number: J00241), CK2 (IMGT accession number: M11736), CK3 (IMGT accession number: M11737), CK4 (IMGT accession number: AF017732) and CK5 (IMGT accession number: AF113887).

In certain embodiments, the C1 comprises an engineered CH1 domain selecting from the group consisting of IgG1, IgG2, IgG3, IgG4, IgM, IgA1, IgA2, IgD, and IgE; and the C2 comprises an engineered λ or κ light chain constant domain (Cλ domain or Cκ domain) from human immunoglobulin, the Cλ domain is selecting from the group consisting of Cλ1, Cλ2, Cλ3, Cλ6 and Cλ7.

In certain embodiments, the C1 comprises an engineered λ or κ light chain constant domain (Cλ domain or Cκ domain) from human immunoglobulin, the Cλ domain is selecting from the group consisting of Cλ1, Cλ2, Cλ3, Cλ6 and Cλ7, and the C2 comprises an engineered CH1 domain selecting from the group consisting of IgG1, IgG2, IgG3, IgG4, IgM, IgA1, IgA2, IgD, and IgE.

In certain embodiments, a) C1 comprises an engineered CH1 domain from human immunoglobulin G1 (IgG1), and C2 comprises an engineered Cλ1 domain from human immunoglobulin; b) C1 comprises an engineered CH1 domain from human immunoglobulin G2 (IgG2), and C2 comprises an engineered Cλ1 domain from human immunoglobulin; c) C1 comprises an engineered CH1 domain from human immunoglobulin G3 (IgG3), and C2 comprises an engineered Cλ1 domain from human immunoglobulin; d) C1 comprises an engineered CH1 domain from human immunoglobulin G4 (IgG4), and C2 comprises an engineered Cλ1 domain from human immunoglobulin; e) C1 comprises an engineered CH1 domain from human immunoglobulin G1 (IgG1), and C2 comprises an engineered Cλ2 domain from human immunoglobulin; f) C1 comprises an engineered CH1 domain from human immunoglobulin G2 (IgG2), and C2 comprises an engineered Cλ2 domain from human immunoglobulin; g) C1 comprises an engineered CH1 domain from human immunoglobulin G3 (IgG3), and C2 comprises an engineered Cλ2 domain from human immunoglobulin; h) C1 comprises an engineered CH1 domain from human immunoglobulin G4 (IgG4), and C2 comprises an engineered Cλ2 domain from human immunoglobulin; i) C1 comprises an engineered CH1 domain from human immunoglobulin G1 (IgG1), and C2 comprises an engineered Cλ1 domain from human immunoglobulin; j) C1 comprises an engineered CH1 domain from human immunoglobulin G2 (IgG2), and C2 comprises an engineered Cλ3 domain from human immunoglobulin; k) C1 comprises an engineered CH1 domain from human immunoglobulin G3 (IgG3), and C2 comprises an engineered Cλ3 domain from human immunoglobulin; l) C1 comprises an engineered CH1 domain from human immunoglobulin G4 (IgG4), and C2 comprises an engineered Cλ3 domain from human immunoglobulin; m) C1 comprises an engineered CH1 domain from human immunoglobulin G1 (IgG1), and C2 comprises an engineered Cλ6 domain from human immunoglobulin; n) C1 comprises an engineered CH1 domain from human immunoglobulin G2 (IgG2), and C2 comprises an engineered Cλ6 domain from human immunoglobulin; o) C1 comprises an engineered CH1 domain from human immunoglobulin G3 (IgG3), and C2 comprises an engineered Cλ6 domain from human immunoglobulin; p) C1 comprises an engineered CH1 domain from human immunoglobulin G4 (IgG4), and C2 comprises an engineered Cλ6 domain from human immunoglobulin; q) C1 comprises an engineered CH1 domain from human immunoglobulin G1 (IgG1), and C2 comprises an engineered Cλ7 domain from human immunoglobulin; r) C1 comprises an engineered CH1 domain from human immunoglobulin G2 (IgG2), and C2 comprises an engineered Cλ7 domain from human immunoglobulin; s) C1 comprises an engineered CH1 domain from human immunoglobulin G3 (IgG3), and C2 comprises an engineered Cλ7 domain from human immunoglobulin; t) C1 comprises an engineered CH1 domain from human immunoglobulin G4 (IgG4), and C2 comprises an engineered Cλ7 domain from human immunoglobulin.

In certain embodiments, the C1 comprises an engineered CH1 from any one of SEQ ID Nos:11, 13, 15, and 17, and/or the C2 comprises an engineered Cλ from any one of SEQ ID Nos:1, 3, 5, 7 and 9.

In certain embodiments, a first Vα is operably linked to C1 through a first conjunction domain, and a first Vβ is operably linked to C2 through a second conjunction domain.

In certain embodiments, the C1 comprises the engineered CH1, and the C2 comprises the engineered Cλ; and wherein the first conjunction domain comprises any one of SEQ ID Nos:19, 21, and 23, and/or the second conjunction domain comprises any one of SEQ ID Nos:25, 27, 29, 31, 33 and 35, preferably, the second conjunction domain comprises EDLXNVXP, the X is any amino acid.

In certain embodiments, the TCR VB comprises mutagenesis at one or more positions selected from 10, 13, 19, 24, 48, 54, 77, 90, 91, 123, and 125 (IMGT numbering) in framework region, preferably, the TCR Vβ comprises at least one mutation at position 13, or comprises at least two mutations at position 90 and 91.

In certain embodiments, the Cλ or CH1 comprises mutagenesis at one or more positions selected from 30, 31 and 33.

In another aspect, the resent disclosure provides a multispecific antigen-binding complex, comprising a first antigen-binding moiety comprising aforesaid polypeptide complex and a second antigen-binding moiety, wherein the first antigen-binding moiety has a first antigenic specificity.

In certain embodiments, the second antigen-binding moiety binds to different epitopes on the first antigen or has a second antigenic specificity which is preferably different from the first antigenic specificity, conjugated at N-terminus or C-terminus of first polypeptide of the first antigen-binding moiety or second polypeptide of the first antigen-binding moiety.

In certain embodiments, the first antigenic specificity and the second antigenic specificity are directed to two different antigens or are directed to two different epitopes on one antigen.

In certain embodiments, the multispecific antigen-binding complex comprises a first antigen binding moiety and a second antigen-binding moiety, wherein the first antigen-binding moiety comprising a first polypeptide comprising, from N-terminus to C-terminus, a first TCR α chain variable domain of a first TCR operably linked to the first antibody constant domain (C1), and a second polypeptide comprising, from N-terminus to C-terminus, a first TCR β chain variable domain of a first TCR operably linked to the second antibody constant domain (C2), wherein C1 and C2 are capable of forming a dimer via it native interchain bond and interactions. The first TCR has a first antigenic specificity.

In certain embodiments, the second antigen-binding moiety has the specificity to different epitopes on the first antigen.

In certain embodiments, the second antigen-binding moiety has a second antigenic specificity which is different from the first antigenic specific, conjugated at N-terminus or C-terminus of first polypeptide or second polypeptide of the first antigen binding moiety or second polypeptide of the first antigen binding moiety.

In certain embodiments, one of the first and the second antigenic specificities is directed to a T-cell specific receptor molecule and/or a natural killer cell (NK cell) specific receptor molecule, and the other is directed to a tumor associated antigen and/or tumor neoantigen.

In certain embodiments, the first antigen-binding moiety comprises a TCR VC and a TCR VR, the Vα comprises an amino acid sequence selected from SEQ ID Nos:37, 41, and 45, the VR comprises an amino acid sequence selected from SEQ ID Nos:39, 43, and 47; preferably, the second antigen-binding moiety comprises a scFv which is selected from SEQ ID No:49.

In certain embodiments, the first antigen-binding moiety binds to HLA*A*02:01-NY-ESO-1 peptide (SLLMWITQC) (SEQ ID Nos:37-40, 45-48), the second antigen-binding moiety binds to cluster of differentiation 3 (CD3) (SEQ ID Nos:49-50).

In certain embodiments, the first antigen-binding moiety binds to HLA*A*02:01-GP100 peptide (YLEPGPVTV) (SEQ ID Nos:41-44), the second antigen-binding moiety binds to CD3 (SEQ ID Nos:49-50).

In certain embodiments, the second antigen-binding moiety comprises a single-chain fragment viable (scFv) containing both heavy chain variable domain and a light chain variable domain covalently conjugated via flexible linker.

In another aspect, the present disclosure provides herein an isolated polynucleotide encoding the polypeptide complex provided herein, or the multispecific antigen-binding complex provided herein.

In one aspect, the present disclosure provides herein an isolated vector comprising the polynucleotide provided herein.

In one aspect, the present disclosure provides herein a host cell comprising the isolated polynucleotide provided herein or the isolated vector provided herein.

In one aspect, the present disclosure provides herein a conjugate comprising the polypeptide complex or the multispecific antigen-binding complex provided herein.

In one aspect, the present disclosure provides herein a method of expressing the polypeptide complex provided herein, or the multispecific antigen-binding complex provided herein, comprising culturing the host cell provided herein under the condition at which the polypeptide complex, or the multispecific antigen-binding complex is expressed.

In one aspect, the present disclosure provides herein a method of producing the polypeptide complex provided herein, comprising a) introducing to a host cell a first polynucleotide encoding a first polypeptide comprising, from N-terminus to C-terminus, a first TCR α chain variable domain of a first TCR operably linked to the first antibody constant domain (C1), and a second polypeptide comprising, from N-terminus to C-terminus, a first TCR β chain variable domain of a first TCR operably linked to the second antibody constant domain (C2), wherein C1 and C2 are capable of forming a dimer via its native inter-chain bonds and interactions. The first TCR has a first antigenic specificity. b) allowing the host cell to express the polypeptide complex.

In one aspect, the present disclosure provides herein a method of producing the multispecific antigen-binding complex provided herein, comprising a) introducing to a host cell a first polynucleotide encoding a first polypeptide comprising, from N-terminus to C-terminus, a first TCR α chain variable domain of a first TCR operably linked to the first antibody constant domain (C1), and a second polypeptide comprising, from N-terminus to C-terminus, a first TCR β chain variable domain of a first TCR operably linked to the second antibody constant domain (C2), wherein C1 and C2 are capable of forming a dimer via its native inter-chain bonds and interactions. The first TCR has a first antigenic specificity. A second antigen-binding moiety has a second antigenic specificity which is different from the first antigenic specific, conjugated at N-terminus or C-terminus of first polypeptide or second polypeptide of the first antigen binding moiety or second polypeptide of the first antigen binding moiety. b) allowing the host cell to express the multispecific antigen-binding complex.

In certain embodiments, the method of producing the multispecific antigen-binding complex provided herein further comprising isolating the polypeptide complex.

In one aspect, the present disclosure provides a composition comprising the polypeptide complex provided herein, or the multispecific antigen-binding complex provided herein.

In one aspect, the present disclosure provides herein a pharmaceutical composition comprising the polypeptide complex provided herein, or the multispecific antigen-binding complex provided herein and a pharmaceutically acceptable carrier.

In one aspect, the present disclosure provides herein a method of treating a condition or disease e.g. cancer in a subject in need thereof, comprising administrating to the subject a therapeutically effective amount of the polypeptide complex provided herein, or the multispecific antigen-binding complex provided herein. In certain embodiments, the condition can be alleviated, eliminated, treated, or prevented when the first antigen and the second antigen are both modulated.

In another aspect, the present disclosure provides a kit comprising the polypeptide complex provided herein for detection, diagnosis, prognosis or treatment of a disease or condition.

### BRIEF DESCRIPTION OF THE DRAWINGS

FIG. 1 shows a schematic diagram of the truncated and mutated amino acid positions in the constant region of the antibody in step 1 of Example 1.

FIG. 2 shows the display and binding activity assay results of the phage-displayable chimeric TCR intermediates obtained from the screening in step 2 of Example 1.

FIG. 3 shows the results of the affinity comparison of the chimeric TCR intermediates obtained from the screening in step 2 of Example 1 with dsTCR and scTCR.

FIG. 4 shows a schematic diagram of the VTCRC IgG1, IgG 4, IgA1 and VTCRCκ, λ interlinker incorporation positions in step 3 of Example 1.

FIG. 5 shows the comparative results of the chimeric TCR phage linker in step 4 of Example 1.

FIG. 6 shows the results of the phage display and binding activity assay of the cloned phage during 1G4 affinity maturation in Example 1 step 5.

FIG. 7A shows modeled structure of representative CTCR, artificial disulfide bond is described as spheres. FIG. 7B shows modeled structure of representative ETCR. The long FG loop, helped to stabilize the Vβ in CTCR was absent in ETCR, resulted a less stable β chain structure.

FIG. 8A shows the conjunction domain structure of CTCR, black arrow indicated the terminus of the conjunction domain analyzed from structure, red dash arrow indicated potential polar contact formed between conjunction domain and FG loop. FIG. 8B shows the conjunction domain structure of ETCR (SSAS), black arrow indicated the terminus of conjunction domain analyzed from superimpose of CTCR and ETCR.

FIG. 9 shows the modeled structure of representative CTCR, the residues involved in variable domain and constant domain binding interface were showed as sticks covered by gray mesh.

FIG. 10A shows the detailed structure of Vβ-Cβ binding interface of representative CTCR, residues involved in binding were showed as sticks, red arrow and yellow dash line indicated polar contact, orange circle indicated non-polar contact. FIG. 10B shows the detailed structure of VR-Cλ binding interface of representative ETCR, residues involved in binding were showed as sticks, red arrow indicated absent polar contact.

FIG. 11A shows the SDS-PAGE result of representative ETCRs. FIG. 11B shows the KD ELISA result of representative ETCRs.

FIG. 12A shows the superimpose result of ETCR1 (cyan) and ETCR2 (magentas), the residues in FR1 were showed as sticks. FIG. 12B shows the SDS-PAGE result of representative ETCR2 mutants.

FIG. 13A-E shows the sensorgrams of SPR analysis of representative ETCRs and CTCRs.

FIG. 14 shows the FACS result of representative ETCR1 and CTCR1.

FIG. 15 presents schematic representations of studied bispecific ETCR. The gene product of anti-CD3 scFv was amplified and inserted at N terminus of TCR Vβ domain, C terminus of antibody Cλ domain, N terminus of TCR Vα domain, C terminus of antibody CH1 domain, respectively, generating bispecific ETCR1-E1.1, ETCR1-E1.2, ETCR1-E1.3 and ETCR1-E1.4 (FIG. 15A-D). For CTCR, The gene product of anti-CD3 scFv was amplified and inserted at N terminus of TCR Vβ domain, generating CTCR1-E1.1 (FIG. 15E).

FIG. 16 shows the SDS-PAGE result of ETCR1 bispecifics, lane 1-4: supernatant of ETCR1 bispecifics, lane 5-8, corresponding purified ETCR1 bispecifics.

FIG. 17A shows the dose-dependent result of redirect T cell killing on T2 cells at 18 h. FIG. 17B shows the dose-dependent result of redirect T cell killing on T2 cells at 24 h.

FIG. 18 shows the dose-dependent result of redirect T cell killing on A375 cells at 72 h.

FIG. 19 shows the formats of ETCRs.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

The term “isolated”, as used herein, refers to a state obtained from natural state by artificial means. A certain “isolated” substance or component may be present in nature possibly because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “vector”, as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including but not limited to plasmids, phages, cosmids, artificial chromosome such as yeast artificial chromosome (YAC), bacterial artificial chromosome (BAC) or P1-derived artificial chromosome (PAC), phage such as λ phage or M13 phage and animal virus. The animal viruses that can be used as vectors, including but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell”, as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but is still included within the scope of the term “host cell.”

The term “SPR” or “surface plasmon resonance”, as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “cancer”, as used herein, refers to any one of a tumor or a malignant cell growth, proliferation or metastasis-mediated, solid tumors and non-solid tumors such as leukemia and initiate a medical condition.

The term “treatment”, “treating”, or “treated”, as used herein in the context of treating a condition, pertain generally to treatment and therapy, whether of a human or an animal, in which some desired therapeutic effect is achieved, for example, the inhibition of the progress of the condition, and include a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount”, or “a therapeutically-effective amount”, as used herein, pertains to the amount of an active compound, or a material, composition or dosage for comprising an active compound, which is effective for producing some desired therapeutic effect, commensurate with a reasonable benefit/risk ratio, when administered in accordance with a desired treatment regimen. For instance, “an effective amount”, when used in connection with treatment of target antigen-related diseases or conditions, refers to an antibody or antigen-binding portion thereof in an amount or concentration effective to treat the said diseases or conditions.

The term “pharmaceutically acceptable”, as used herein, means that the vehicle, diluent, excipient and/or salts thereof, are chemically and/or physically compatible with other ingredients in the formulation, and physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except where otherwise noted, the terms “patient” or “subject” are used interchangeably.

The experimental methods in the following examples are conventional methods, unless otherwise specified.

### EXAMPLES

### Example 1. Gene of TCR Variable Domain is Ligated with the Gene of Constant Region of the Antibody and Further Assembled with the Gill Gene of the Phage for Screening the TCR Heterodimer Format that can be Displayed on the Surface of the Phage

Step 1. Genes of Two Different TCR V Domain: CTCR2 and CTCR2 is Ligated with the Gene of Antibody Constant Region.

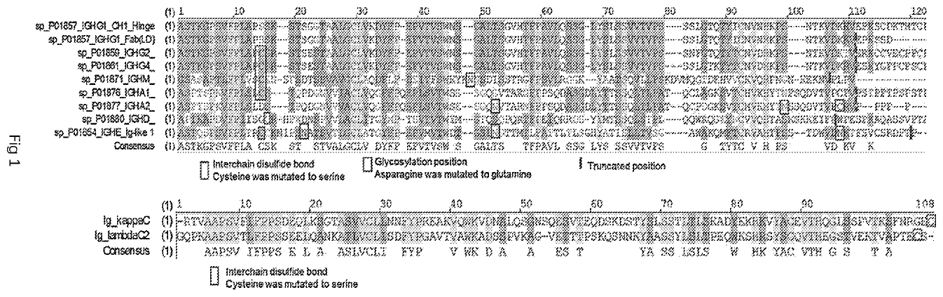

The heavy chain constant region of an antibody includes the CH1 domain or full length of IgG1, IgG2, IgG4, IgM, IgA1, IgA2, IgD, IgE and the light chain constant region CK, Cλ of the antibody. Particularly, two extra mutations in antibody constant domain were designed, tested and compared with wildtype, 1). interchain disulfide bond mutation: cysteine to serine, (if the C of the interchain disulfide bond position in the constant region of the antibody is mutated to S, then the constant region CH1 will be marked as: κG1s, κG1s-Reverse) 2). N-glycosylation mutation: asparagine to glutamine. The truncation and mutation positions are shown in FIG. 1. The ligation resulted V_{TCR}C_{IgG1, IgG 2, IgG4, IgA1, IgA2, IgM, IgD, IgE} and V_{TCR}C_{κ,λ} gene products (V_{TCR} includes Vα and Vβ). These gene products are inserted into the phagemid vector pcom3XX-DT, where V_{TCR}C_{IgG1, IgG 2, IgG4, IgA1, IgA2, IgM, IgD, IgE} V_{TCR}C_{κ,λ} are conjugated with gill gene expressing the c-Myc.6His tag and V_{TCR}C_{κ,λ} is expressing Flag tag. Since the phage can self-assemble, the two expressed polypeptides are spontaneously combined and displayed as a functional heterodimer on the phage. The corresponding CTCR is also inserted into the same phagemid vector where VRCβ are conjugated with gill gene expressing the c-Myc.6His tag and VαCα is expressing Flag tag. The corresponding single chain TCR is assembled as Vα-(G4S)-Vβ and inserted into the plasmid vector pFL249 fused with gill gene expressing 6His.c-Myc tag.

### Step 2. Optimization of Phage Culture Conditions.

For screening the phages displaying different TCR heterodimers format, clones were inoculated into 600 μl 2YT medium (10 g/L yeast extract, 16 g/L trypsin, 5 g/ml containing 0.1 mg/ml ampicillin and 2% glucose. pH 7.0). The strain was grown in 37° C. shaker to OD₆₀₀=0.3 to 0.5, and 7.5E9 pfu helper phage M13KO7 (Invitrogen) was added, and further incubated at 37° C. incubator for 45 minutes. The cells were pelleted by centrifugation at 4,000 g, 10 mins, resuspended in 600 μl 2YT medium containing 0.1 mg/ml ampicillin and 0.05 kanamycin, 5 mM MgSO₄ and cultured in 25° C. shaker for 36 hours. Phage supernatant displaying the TCR heterodimer can be obtained via centrifugation.

### Step 3. Phage ELISA Detects the Phage Display and Binding Activity of TCR Heterodimer.

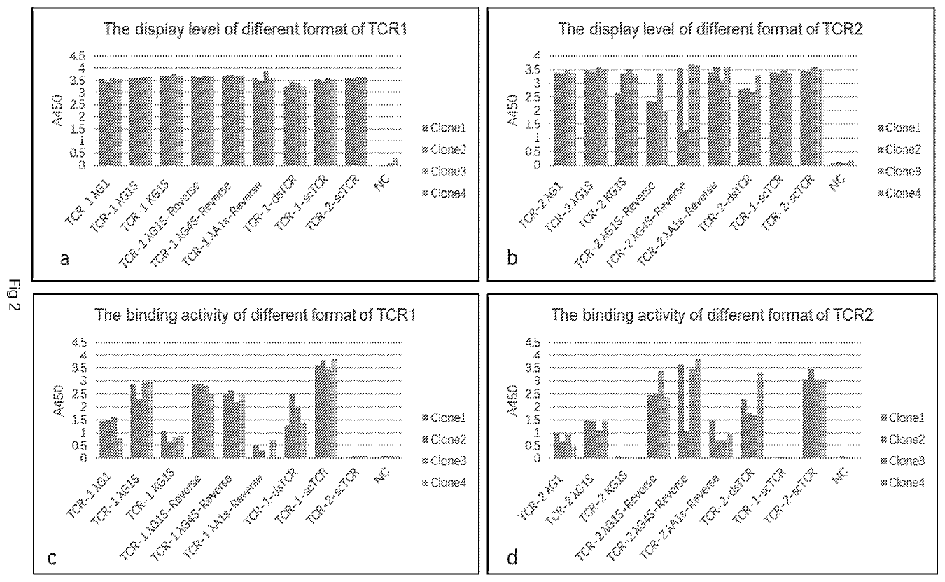

The display level of TCR heterodimer on phage was detected by the sandwich ELISA method. The ELISA plate was coated with anti-Flag (2 μg/ml) to capture the ETCR or CTCR heterodimer phage, and coated with anti-c-myc (1 μg/ml) to capture the scTCR. Block the plates with blocking solution (3% BSA) for 1 hour. 1:1 diluted phage supernatant was added and incubate at room temperature (20-25° C.) for 2 hours. Wash 6 times with 1×PBST, then anti-phage M13 alkaline phosphatase conjugated antibody was used to detect the display level of TCR on the surface of phage. The binding performance TCR heterodimer on phage was detected by direct ELISA method. The ELISA plate was coated with 4 μg/ml SA overnight and blocked with blocking solution (3% BSA) for 1 hour. 2 μg/ml biotinylated pMHCI monomer was added and incubated at RT for 1 hour. Wash 6 times with 1×PBST, and then add 1:1 diluted phage supernatant and incubated at RT for 1 hour, then anti-phage M13 alkaline phosphatase-conjugated antibody was used to detect the binding activity of TCR-displayed phage. Finally, 64 ETCR format was screened and 6 optimal formats were obtained: λG1, λG1s, κG1s, λG1s-Reverse, λG4s-Reverse, λA1s-Reverse, which revealed better display level and binding performance, as shows in FIG. 2.

FIG. 2A and FIG. 2B shows the display level of TCR1 and TCR2 in different TCR format respectively. Four clones were randomly selected for each displayed TCR for testing. The results suggested that all ETCR, CTCR and scTCR can be displayed well by phage.

FIG. 2C and FIG. 2D shows the binding performance of TCR1 and TCR2 in different TCR format respectively. Different binding to specific pMHCI was observed with different TCR format, where no non-specific binding to pMHCI was observed.

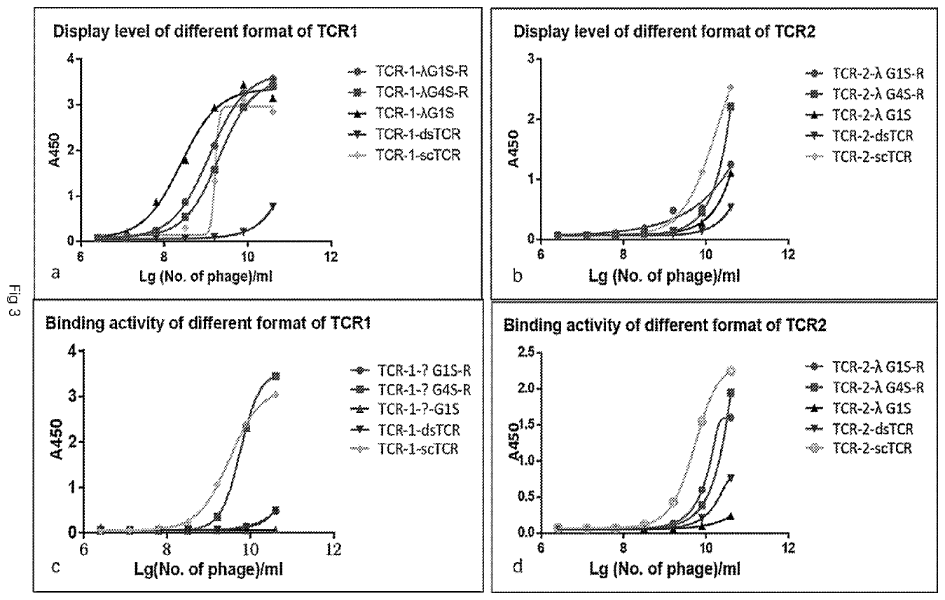

Display level and binding activity of the optimal ETCR format was further determined and compared with CTCR and scTCR by phage relative quantitative ELISA. The number of phage in the initial well was 5E10 pfu, with a dilution of 1:3. The results show that the display level of optimal ETCR format is better than scTCR and dsTCR. Also, as shown in the FIG. 3, the binding affinity of our TCR λG4s-Reverse format is 2-6 times better than that of dsTCR. For further optimizing the ETCR format, linker domain was subsequently designed.

### Step 4. Linker Optimization of the Chimeric TCR Structure.

In order to improve the display level and binding activity of the ETCR, FR4 part of TCR Vα or Vβ was truncated, and then directly connect to the constant domain of the antibody. The results showed that the stability of the TCR was affected and the binding activity was reduced.

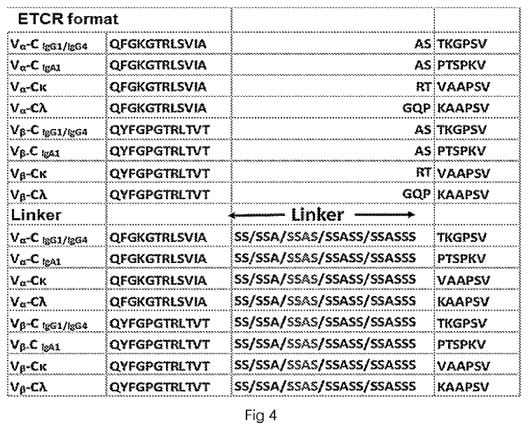

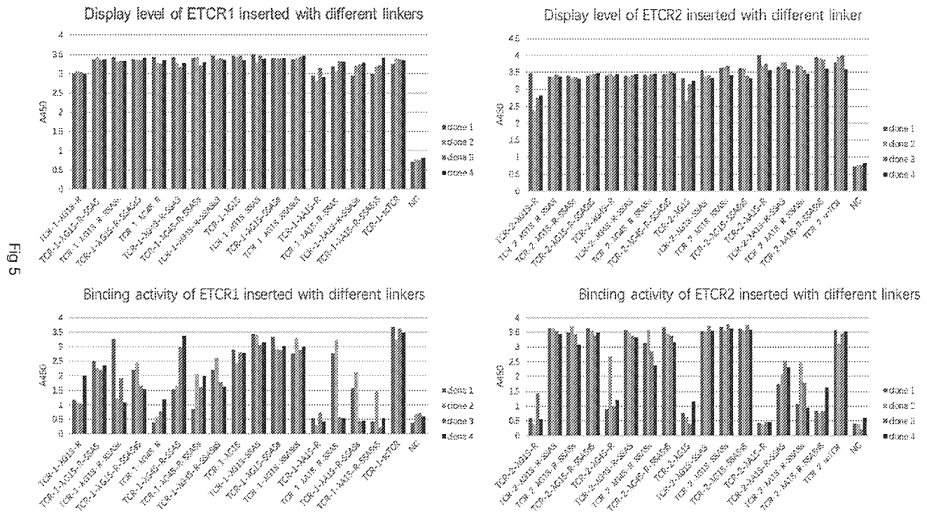

On the other hand, linkers of different lengths including SS, SSA, SSAS, SSASS, SSASSS were inserted between the C-terminus of the ETCR variable domain and the N-terminus of the antibody constant domain, the specific location is shown in FIG. 4. Four clones are randomly selected for the detection of phage display level and binding activity. Among all linkers, SSAS linker showed superior performance than the other linkers (FIG. 5). Thus, λG4s-Reverse-SSAS was selected as the final ETCR format for TCR affinity maturation.

### Step 5. Construction and Screening of TCR Affinity Maturation Library.

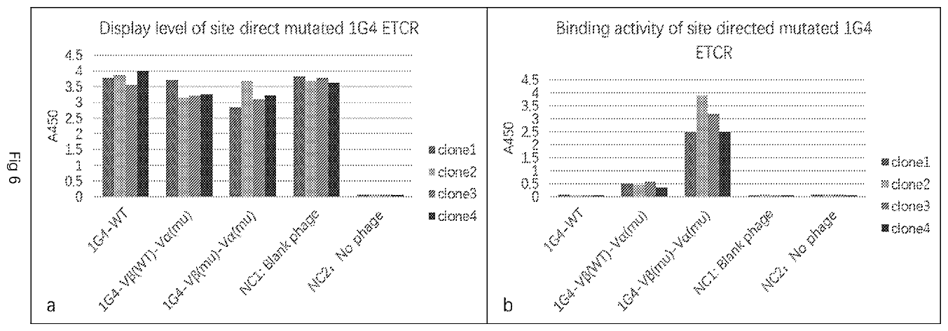

Native TCR 1G4 was selected for affinity maturation as Proof-of-Concept study. Vα and Vβ genes of 1G4 was synthezed and cloned into our phagemid vector comprising λG4s-Reverse-SSAS ETCR backbone, resulted 1G4 ETCR (template for affinity maturation, wildtype, WT). The affinity of native TCR is extremely low that no binding signal can be detected using the ELISA. Subsequently, we made site directed mutations on the CDR2 and CDR3 of 1G4 ETCR according to reference, as shown in FIG. 6, binding activity was detected, proved the feasibility of TCR affinity maturation using ETCR heterodimer format.

### Example 2: Shuffling of TCR Viable Domain with Antibody Constant Domain for Generating TCR-Antibody Chimeric Proteins (ETCR)

### 1. TCR Sequence

An HLA*A*02:01 NY-ESO-1 (SLLMWITQC) specific TCR, with a non-native disulfide bond between Cα S48-Cβ T57, named CTCR1 (SEQ ID No:37-40, and 63-66, the amino acid sequence of Vα is shown as SEQ ID No:37, the amino acid sequence of Vβ is shown as SEQ ID No:39, the amino acid sequence of Cα is shown as SEQ ID No:63, the amino acid sequence of Vβ is shown as SEQ ID No:65), and an HLA*A*02:01 GP100 (YLEPGPVTV) specific TCR, with a non-native disulfide bond between Cα S48-CR T57, named CTCR2 (SEQ ID No:41-44, and 63-66, the amino acid sequence of Vα is shown as SEQ ID No:41, the amino acid sequence of Vβ is shown as SEQ ID No:43, the amino acid sequence of Cα is shown as SEQ ID No:63, the amino acid sequence of Vβ is shown as SEQ ID No:65), were selected to conduct the Proof-of-Concept study. IMGT numbering rule was used for all TCR variable domains.

Another HLA*A*02:01 NY-ESO-1 (SLLMWITQC) specific TCR, with a non-native disulfide bond between Cα S48-CR T57, named CTCR3 (SEQ ID Nos:45-48, and 63-66, the amino acid sequence of Vα is shown as SEQ ID No:45, the amino acid sequence of Vβ is shown as SEQ ID No:47, the amino acid sequence of Cα is shown as SEQ ID No:63, the amino acid sequence of Vβ is shown as SEQ ID No:65), were as well selected to conduct the further study. IMGT numbering rule was used for all TCR variable domains.

SEQ ID No: 37, CAb1-NY-ESO-1_Vα AA:
AQSVAQPEDQVNVAEGNPLTVKCTYSVSGNPYLFWYVQYPNRGLQFLLKYLGDSALVKGSYGFEAEFNKSQTSFHLKK
PSALVSDSALYFCAVRDIRSGAGSYQLTFGKGTKLSVIP
SEQ ID No: 38, CAb1-NY-ESO-1_Vα DNA:
gcccagtccgtggctcagcccgaggaccaagtgaacgtggccgagggcaaccctctgaccgtgaagtgcacctattccgtgagcggcaacccctat
ctgttttggtacgtgcagtaccccaacagaggactgcagtttctgctgaagtatctgggagacagcgctctggtgaagggaagctacggcttcgaagc
cgagttcaacaagagccagacctccttccatctgaagaagcctagcgctctggtgagcgactccgctctgtacttctgcgccgtcagagacatcagaa
gcggcgccggaagctaccagctgaccttcggcaagggcaccaagctgagcgtgatccct
SEQ ID No: 39, CAb1-NY-ESO-1_Vβ AA:
SAVISQKPSRDIKQRGTSLTIQCQVDKRLALMFWYRQQPGQSPTLIATAWTGGEATYESGFVIDKFPISRPNLTFSTLTVS
NMSPEDSSIYLCSVGGSGAADTQYFGPGTRLTVL
SEQ ID No: 40, CAb1-NY-ESO-1_Vβ DNA:
agcgccgtgatcagccagaagcctagcagagacatcaaacagaggggcacatctctgaccatccagtgccaagtggacaagagactcgctctgatg
ttctggtatagacagcagcccggacagtcccccacactgatcgccaccgcttggaccggcggagaagccacctacgagtccggcttcgtgatcgaca
agttccccatctctagacccaatctgaccttttccacactgaccgtgtccaacatgagccccgaggactccagcatttatctgtgtagcgtgggaggcag
cggagctgccgatacccagtacttcggccccggaaccagactgaccgtgctg
SEQ ID No: 41, CAb2-GP100_Vα AA:
AQQGEEDPQALSIQEGENATMNCSYKTSINNLQWYRQNSGRGLVHLILIRSNEREKHSGRLRVTLDTSKKSSSLLITASR
AADTASYFCATDGSTPMQFGKGTRLSVIA
SEQ ID No: 42, CAb2-GP100_Vα DNA:
gctcagcaaggcgaagaggatccccaagctctgagcattcaagagggcgagaacgccaccatgaactgctcctacaagaccagcatcaacaacctc
cagtggtatagacagaacagcggcagaggactggtgcatctgattctgattagaagcaacgagagagagaagcactccggaaggctgagggtga
cactggatacaagcaagaagagcagctctctgctgatcaccgcttccagagccgctgacaccgccagctacttctgcgccaccgacggcagcacccct
atgcagttcggcaagggcacaagactcagcgtgatcgcc
SEQ ID No: 43, CAb2-GP100_Vβ AA:
DGGITQSPKYLFRKEGQNVTLSCEQNLNHDAMYWYRQDPGQGLRLIYYSWAQGDFQKGDIAEGYSVSREKKESFPLT
VTSAQKNPTAFYLCASSWGAPYEQYFGPGTRLTVT
SEQ ID No: 44, CAb2-GP100_Vβ DNA:
gacggcggcatcacccagtcccccaagtatctgtttagaaaggagggccagaatgtgacactgagctgcgagcagaatctgaaccacgacgccatg
tactggtacagacaagaccccggccaaggactgaggctgatctattacagctgggcacaaggagacttccagaagggcgacatcgccgagggata
cagcgtgtctagagagaagaaggagagctttcctctgaccgtgaccagcgcccagaagaatcccaccgccttctatctgtgtgccagcagctgggga
gctccctacgagcagtatttcggacccggcacaagactgaccgtgaca
SEQ ID No: 45, CAb3-NY-ESO-1_Vα AA:
QEVTQIPAALSVPEGENLVLNCSFTDSAIYNLQWFRQDPGKGLTSLLLITPWQREQTSGRLNASLDKSSGRSTLYIAASQ
PGDSATYLCAVRPLLDGTYIPTFGRGTSLIVHP
SEQ ID No: 46, CAb3-NY-ESO-1_Vα DNA:
caagaagtgacacagatccctgccgctctgtctgtgcctgagggcgaaaacctggtgctgaactgcagcttcaccgacagcgccatctacaacctgca
gtggttcagacaggaccccggcaagggactgacaagcctgctgctgattaccccttggcagagagagcagaccagcggcagactgaatgccagcc
tggataagtcctccggcagaagcaccctgtatatcgccgcttctcagcctggcgatagcgccacatatctgtgtgccgtcagacccctgctggacggca
catatatccccacctttggcagaggcaccagcctgatcgtgcaccct
SEQ ID No: 47, CAb3-NY-ESO-1_Vβ AA:
GVTQTPKFQVLKTGQSMTLQCAQDMNHEYMSWYRQDPGMGLRLIHYSVAIQTTDRGEVPNGYNVSRSTIEDFPLRLL
SAAPSQTSVYFCASSYLGNTGELFFGEGSRLTVL
SEQ ID No: 48, CAb3-NY-ESO-1_Vβ DNA:
ggagttacacagacccctaagttccaggtgctgaaaaccggccagagcatgaccctgcagtgcgcccaggatatgaaccacgagtacatgagctgg
tacaggcaggatccaggcatgggcctgagactgatccactactctgtggccatccagaccaccgacagaggcgaagtgcccaacggctacaacgtg
tccagatccaccatcgaggacttcccactgagactgctgtctgctgcccctagccagacctccgtgtacttttgtgccagcagctacctgggcaacaccg
gcgagctgttttttggcgagggctccagactgaccgtgctg
SEQ ID No: 49, Anti-CD3-scFv AA:
AIQMTQSPSSLSASVGDRVTITCRASQDIRNYLNWYQQKPGKAPKLLIYYTSRLESGVPSRFSGSGSGTDYTLTISSLQPE
DFATYYCQQGNTLPWTFGQGTKVEIKGGGGSGGGGSGGGGSGGGGSGGGSEVQLVESGGGLVQPGGSLRLSCAAS
GYSFTGYTMNWVRQAPGKGLEWVALINPYKGVSTYNQKFKDRFTISVDKSKNTAYLQMNSLRAEDTAVYYCARSGYY
GDSDWYFDVWGQGTLVTVSS
SEQ ID No: 50, Anti-CD3-scFv DNA:
gccatccagatgacgcaaagtccatcaagtctgagcgccagcgtgggcgacagagtgaccatcacctgcagagccagccaggacatcagaaattac
ctgaattggtaccagcagaagcctggcaaggctccaaagctcctcatatattatacatcgagattagaatctggtgttccaagcagattcagcggcagc
ggcagcggcaccgactacaccctgaccatcagcagcctgcagcctgaggacttcgccacctactactgccagcagggcaataccctgccttggacatt
tggacagggtaccaaggtggaaattaaaggcggcggcggaagcggaggcggagggtcgggggcggaggttcaggtggaggagggtctggt
ggaggctcagaggtacaacttgtggagtcaggcggtggactagtccaaccaggaggatctttacgcttatcttgtgccgccagcggctacagcttcac
cggctacaccatgaattgggtgagacaggctcccggtaagggcctggagtgggtggccctgatcaatccttacaagggcgtgagcacctacaatca
gaagttcaaggacagattcaccatcagcgtggacaagagcaagaataccgcctacctgcagatgaatagcctgagagccgaggacaccgccgtgt
actactgcgccagaagcggctactacggcgacagcgactggtactttgatgtttgggggcaaggtacacttgtcactgtaagctcc
SEQ ID No: 51, CAb1-NY-ESO-1_αFL AA:
AQSVAQPEDQVNVAEGNPLTVKCTYSVSGNPYLFWYVQYPNRGLQFLLKYLGDSALVKGSYGFEAEFNKSQTSFHLKK
PSALVSDSALYFCAVRDIRSGAGSYQLTFGKGTKLSVIPNIQNPDPAVYQLRDSKSSDKSVCLFTDFDSQTNVSQSKDSD
VYITDKCVLDMRSMDFKSNSAVAWSNKSDFACANAFNNSIIPEDT
SEQ ID No: 52, CAb1-NY-ESO-1_αFL DNA:
gcccagtccgtggctcagcccgaggaccaagtgaacgtggccgagggcaaccctctgaccgtgaagtgcacctattccgtgagcggcaacccctat
ctgttttggtacgtgcagtaccccaacagaggactgcagtttctgctgaagtatctgggagacagcgctctggtgaagggaagctacggcttcgaagc
cgagttcaacaagagccagacctccttccatctgaagaagcctagcgctctggtgagcgactccgctctgtacttctgcgccgtcagagacatcagaa
gcggcgccggaagctaccagctgaccttcggcaagggcaccaagctgagcgtgatccctaacatccagaaccccgatcccgccgtgtaccagctga
gggacagcaagtccagcgacaagtccgtgtgtctgttcaccgacttcgactcccagaccaacgtgtcccagagcaaggatagcgacgtgtacatcac
cgacaagtgcgtcctcgacatgaggtccatggacttcaagagcaacagcgccgtggcttggagcaacaagagcgacttcgcttgcgccaacgccttc
aacaacagcatcatccccgaggacacc
SEQ ID No: 53, CAb1-NY-ESO-1_βFL AA:
SAVISQKPSRDIKQRGTSLTIQCQVDKRLALMFWYRQQPGQSPTLIATAWTGGEATYESGFVIDKFPISRPNLTFSTLTVS
NMSPEDSSIYLCSVGGSGAADTQYFGPGTRLTVLEDLKNVFPPEVAVFEPSEAEISHTQKATLVCLATGFYPDHVELSW
WVNGKEVHSGVCTDPQPLKEQPALNDSRYALSSRLRVSATFWQDPRNHFRCQVQFYGLSENDEWTQDRAKPVTQIVSAEAWGRAD
SEQ ID No: 54, CAb1-NY-ESO-1_βFL DNA:
agcgccgtgatcagccagaagcctagcagagacatcaaacagaggggcacatctctgaccatccagtgccaagtggacaagagactcgctctgatg
ttctggtatagacagcagcccggacagtcccccacactgatcgccaccgcttggaccggcggagaagccacctacgagtccggcttcgtgatcgaca
agttccccatctctagacccaatctgaccttttccacactgaccgtgtccaacatgagccccgaggactccagcatttatctgtgtagcgtgggaggcag
cggagctgccgatacccagtacttcggccccggaaccagactgaccgtgctggaggatctgaagaacgtgtttccccccgaggtggccgtgtttgag
cccagcgaggccgagattagccacacccagaaggccacactggtgtgtctggccaccggcttttaccccgaccacgtggaactgagctggtgggtg
aacggcaaggaggtgcactccggcgtgtgtaccgatccccagcctctgaaggagcagcccgccctcaacgatagcagatacgctctgtcctccagac
tgagagtgagcgccacattctggcaagaccccagaaaccactttagatgccaagtgcagttctacggactgagcgaaaacgacgagtggacacaag
atagagccaagcccgtgacccagatcgtgagcgccgaggcttggggcagagccgat
SEQ ID No: 55, CAb2-GP100_αFL AA:
AQQGEEDPQALSIQEGENATMNCSYKTSINNLQWYRQNSGRGLVHLILIRSNEREKHSGRLRVTLDTSKKSSSLLITASR
AADTASYFCATDGSTPMQFGKGTRLSVIANIQKPDPAVYQLRDSKSSDKSVCLFTDFDSQTNVSQSKDSDVYITDKCVL
DMRSMDFKSNSAVAWSNKSDFACANAFNNSIIPEDT
SEQ ID No: 56, CAb2-GP100_αFL DNA:
gctcagcaaggcgaagaggatccccaagctctgagcattcaagagggcgagaacgccaccatgaactgctcctacaagaccagcatcaacaacctc
cagtggtatagacagaacagcggcagaggactggtgcatctgattctgattagaagcaacgagagagagaagcactccggaaggctgagggtga
cactggatacaagcaagaagagcagctctctgctgatcaccgcttccagagccgctgacaccgccagctacttctgcgccaccgacggcagcaccoct
atgcagttcggcaagggcacaagactcagcgtgatcgccaacatccagaagcccgaccccgccgtgtaccagctgagagactccaagagcagcga
caagagcgtgtgtctgttcaccgacttcgactcccagaccaacgtgagccagtccaaggacagcgacgtgtacatcaccgacaagtgcgtgctggac
atgaggagcatggacttcaagtccaacagcgccgtggcttggtccaacaaatccgatttcgcttgcgccaatgccttcaacaactccatcatccccgag
gacaca
SEQ ID No: 57, CAb2-GP100_βFL AA:
DGGITQSPKYLFRKEGQNVTLSCEQNLNHDAMYWYRQDPGQGLRLIYYSWAQGDFQKGDIAEGYSVSREKKESFPLT
VTSAQKNPTAFYLCASSWGAPYEQYFGPGTRLTVTEDLKNVFPPEVAVFEPSEAEISHTQKATLVCLATGFYPDHVELSW
WVNGKEVHSGVCTDPQPLKEQPALNDSRYALSSRLRVSATFWQDPRNHFRCQVQFYGLSENDEWTQDRAKPVTQIVSAEAWGRAD
SEQ ID No: 58, CAb2-GP100_βFL DNA:
gacggcggcatcacccagtcccccaagtatctgtttagaaaggagggccagaatgtgacactgagctgcgagcagaatctgaaccacgacgccatg
tactggtacagacaagaccccggccaaggactgaggctgatctattacagctgggcAcaaggagacttccagaagggcgacatcgccgagggata
cagcgtgtctagagagaagaaggagagctttcctctgaccgtgaccagcgcccagaagaatcccaccgccttctatctgtgtgccagcagctgggga
gctccctacgagcagtatttcggacccggcacaagactgaccgtgacagaggatctgaagaacgtcttccctcccgaggtggctgtgttcgagccctc
cgaggccgagatctcccacacccagaaggccaccctcgtgtgtctggctaccggcttctaccccgaccacgtggagctgagctggtgggtgaacgg
caaagaggtgcatagcggcgtgtgtaccgacccccagcctctgaaagagcaacccgctctgaacgactccagatacgctctgtcctccagactgagg
gtctccgccacattttggcaagaccctagaaaccactttagatgtcaagtgcagttctacggactgagcgagaatgatgagtggacacaagacagag
ccaagcccgtgacacagattgtcagcgccgaggcttggggaagagctgat
SEQ ID No: 59, CAb3-NY-ESO-1_Vα AA:
QEVTQIPAALSVPEGENLVLNCSFTDSAIYNLQWFRQDPGKGLTSLLLITPWQREQTSGRLNASLDKSSGRSTLYIAASQ
PGDSATYLCAVRPLLDGTYIPTFGRGTSLIVHP
SEQ ID No: 60, CAb3-NY-ESO-1_Vα DNA:
caagaagtgacacagatccctgccgctctgtctgtgcctgagggcgaaaacctggtgctgaactgcagcttcaccgacagcgccatctacaacctgca
gtggttcagacaggaccccggcaagggactgacaagcctgctgctgattaccccttggcagagagagcagaccagcggcagactgaatgccagcc
tggataagtcctccggcagaagcaccctgtatatcgccgcttctcagcctggcgatagcgccacatatctgtgtgccgtcagacccctgctggacggca
catatatccccacctttggcagaggcaccagcctgatcgtgcaccct
SEQ ID No: 61, CAb3-NY-ESO-1_Vβ AA:
GVTQTPKFQVLKTGQSMTLQCAQDMNHEYMSWYRQDPGMGLRLIHYSVAIQTTDRGEVPNGYNVSRSTIEDFPLRLL
SAAPSQTSVYFCASSYLGNTGELFFGEGSRLTVL
SEQ ID No: 62, CAb3-NY-ESO-1_Vβ DNA:
ggagttacacagacccctaagttccaggtgctgaaaaccggccagagcatgaccctgcagtgcgcccaggatatgaaccacgagtacatgagctgg
tacaggcaggatccaggcatgggcctgagactgatccactactctgtggccatccagaccaccgacagaggcgaagtgcccaacggctacaacgtg
tccagatccaccatcgaggacttcccactgagactgctgtctgctgcccctagccagacctccgtgtacttttgtgccagcagctacctgggcaacaccg
gcgagctgttttttggcgagggctccagactgaccgtgctg
SEQ ID No: 63, Cα AA:
NIQKPDPAVYQLRDSKSSDKSVCLFTDFDSQTNVSQSKDSDVYITDKCVLDMRSMDFKSNSAVAWSNKSDFACANAFNNSIIPEDT
SEQ ID No: 64, Cα DNA:
aacatccagaagcccgaccccgccgtgtaccagctgagagactccaagagcagcgacaagagcgtgtgtctgttcaccgacttcgactcccagacca
acgtgagccagtccaaggacagcgacgtgtacatcaccgacaagtgcgtgctggacatgaggagcatggacttcaagtccaacagcgccgtggctt
ggtccaacaaatccgatttcgcttgcgccaatgccttcaacaactccatcatccccgaggacaca
SEQ ID No: 65, Cβ AA:
EDLKNVFPPEVAVFEPSEAEISHTQKATLVCLATGFYPDHVELSWWVNGKEVHSGVCTDPQPLKEQPALNDSRYALSSR
LRVSATFWQDPRNHFRCQVQFYGLSENDEWTQDRAKPVTQIVSAEAWGRAD
SEQ ID No: 66, Cβ DNA:
gaggatctgaagaacgtcttccctcccgaggtggctgtgttcgagccctccgaggccgagatctcccacacccagaaggccaccctcgtgtgtctggc
taccggcttctaccccgaccacgtggagctgagctggtgggtgaacggcaaagaggtgcatagcggcgtgtgtaccgacccccagcctctgaaaga
gcaacccgctctgaacgactccagatacgctctgtcctccagactgagggtctccgccacattttggcaagaccctagaaaccactttagatgtcaagtg
cagttctacggactgagcgagaatgatgagtggacacaagacagagccaagcccgtgacacagattgtcagcgccgaggcttggggaagagctg
at

### 2. Generating TCR-Antibody Chimeric Proteins (ETCR)

The constant domains Cα and Cβ of CTCR1 and CTCR2 were replaced by the constant domain CH1 and Cλ/Cκ of IgA, IgD, IgE, IgG and IgM antibody, fused with or without Fc domain, generating dozens of ETCRs for further analysis.

SEQ ID No: 1, Engineered Cλ1 AA:
PTVTLFPPSSEELQANKATLVCLISDFYPGAVTVAWKADGSPVKAGVE
TTKPSKQSNNKYAASSYLSLTPEQWKSHRSYSCQVTHEGSTVEKTVAP
TECS
SEQ ID No: 2, Engineered Cλ1 DNA:
cccacggtcactctgttcccgccctcctctgaggagctccaagccaac
aaggccacactagtgtgtctgatcagtgacttctacccgggagctgtg
acagtggcttggaaggcagatggcagccccgtcaaggcgggagtggag
acgaccaaaccctccaaacagagcaacaacaagtacgcggccagcagc
tacctgagcctgacgcccgagcagtggaagtcccacagaagctacagc
tgccaggtcacgcatgaagggagcaccgtggagaagacagtggcccct
acagaatgttca
SEQ ID No: 3, Engineered Cλ2 AA:
PTVTLFPPSSEELQANKATLVCLISDFYPGAVTVAWKADGSPVKAGVE
TTKPSKQSNNKYAASSYLSLTPEQWKSHRSYSCQVTHEGSTVEKTVAP
TECS
SEQ ID No: 4, Engineered Cλ2 DNA:
ccctcggtcactctgttcccgccctcctctgaggagcttcaagccaac
aaggccacactggtgtgtctcataagtgacttctacccgggagccgtg
acagtggcttggaaagcagatagcagccccgtcaaggcgggagtggag
accaccacaccctccaaacaaagcaacaacaagtacgcggccagcagc
tatctgagcctgacgcctgagcagtggaagtcccacagaagctacagc
tgccaggtcacgcatgaagggagcaccgtggagaagacagtggcccct
acagaatgttca
SEQ ID No: 5, Engineered Cλ3 AA:
PSVTLFPPSSEELQANKATLVCLISDFYPGAVTVAWKADSSPVKAGVE
TTTPSKQSNNKYAASSYLSLTPEQWKSHKSYSCQVTHEGSTVEKTVAP
TECS
SEQ ID No: 6, Engineered Cλ3 DNA:
ccctcggtcactctgttcccaccctcctctgaggagcttcaagccaac
aaggccacactggtgtgtctcataagtgacttctacccgggagccgtg
acagttgcctggaaggcagatagcagccccgtcaaggcgggggtggag
accaccacaccctccaaacaaagcaacaacaagtacgcggccagcagc
tacctgagcctgacgcctgagcagtggaagtcccacaaaagctacagc
tgccaggtcacgcatgaagggagcaccgtggagaagacagttgcccct
acggaatgttca
SEQ ID No: 7, Engineered Cλ6 AA:
PSVTLFPPSSEELQANKATLVCLISDFYPGAVKVAWKADGSPVNTGVE
TTTPSKQSNNKYAASSYLSLTPEQWKSHRSYSCQVTHEGSTVEKTVAP
AECS
SEQ ID No: 8, Engineered Cλ6 DNA:
ccatcggtcactctgttcccgccctcctctgaggagcttcaagccaac
aaggccacactggtgtgcctgatcagtgacttctacccgggagctgtg
aaagtggcctggaaggcagatggcagccccgtcaacacgggagtggag
accaccacaccctccaaacagagcaacaacaagtacgcggccagcagc
tacctgagcctgacgcctgagcagtggaagtcccacagaagctacagc
tgccaggtcacgcatgaagggagcaccgtggagaagacagtggcccct
gcagaatgttca
SEQ ID No: 9, Engineered Cλ7 AA:
PSVTLFPPSSEELQANKATLVCLVSDFYPGAVTVAWKADGSPVKVGVE
TTKPSKQSNNKYAASSYLSLTPEQWKSHRSYSCRVTHEGSTVEKTVAP
AECS
SEQ ID No: 10, Engineered Cλ7 DNA:
ccctcggtcactctgttcccaccctcctctgaggagcttcaagccaac
aaggccacactggtgtgtctcgtaagtgacttctacccgggagccgtg
acagtggcctggaaggcagatggcagccccgtcaaggtgggagtggag
accaccaaaccctccaaacaaagcaacaacaagtatgcggccagcagc
tacctgagcctgacgcccgagcagtggaagtcccacagaagctacagc
tgccgggtcacgcatgaagggagcaccgtggagaagacagtggcccct
gcagaatgctct
SEQ ID No: 11, Engineered IgG1 CH1 AA:
TKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
SEQ ID No: 12, Engineered IgG1 CH1 DNA:
accaagggcccatcggtcttccccctggcaccctcctccaagagcacc
tctgggggcacagcggccctgggctgcctggtcaaggactacttcccc
gaaccggtgacggtgtcgtggaactcaggcgccctgaccagcggcgtg
cacaccttcccggctgtcctacagtcctcaggactctactccctcagc
agcgtggtgaccgtgccctccagcagcttgggcacccagacctacatc
tgcaacgtgaatcacaagcccagcaacaccaaggtggacaagaaagtt
g
SEQ ID No: 13, Engineered IgG2 CH1 AA:
TKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTV
SEQ ID No: 14, Engineered IgG2 CH1:
accaagggcccatcggtcttccccctggcgccctgctccaggagcacc
tccgagagcacagccgccctgggctgcctggtcaaggactacttcccc
gaaccggtgacggtgtcgtggaactcaggcgctctgaccagcggcgtg
cacaccttcccagctgtcctacagtcctcaggactctactccctcagc
agcgtggtgaccgtgccctccagcaacttcggcacccagacctacacc
tgcaacgtagatcacaagcccagcaacaccaaggggacaagacagtt
SEQ ID No: 15, Engineered IgG3 CH1 AA:
TKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRV
SEQ ID No: 16, Engineered IgG3 CH1 DNA:
accaagggcccatcggtcttccccctggcgccctgctccaggagcacc
tctgggggcacagcggccctgggctgcctggtcaaggactacttcccc
gaaccggtgacggtgtcgtggaactcaggcgccctgaccagcggcgtg
cacaccttcccggctgtcctacagtcctcaggactctactccctcagc
agcgtggtgaccgtgccctccagcagcttgggcacccagacctacacc
tgcaacgtgaatcacaagcccagcaacaccaaggtggacaagagagtt
SEQ ID No: 17, Engineered IgG4 CH1 AA:
TKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRV
SEQ ID No: 18, Engineered IgG4 CH1 DNA:
accaagggcccatccgtcttccccctggcgccctgctccaggagcacc
tccgagagcacagccgccctgggctgcctggtcaaggactacttcccc
gaaccggtgacggtgtcgtggaactcaggcgccctgaccagcggcgtg
cacaccttcccggctgtcctacagtcctcaggactctactccctcagc
agcgtggtgaccgtgccctccagcagcttgggcacgaagacctacacc
tgcaacgtagatcacaagcccagcaacaccaaggtggacaagagagtt

### 3. Materials and Methods

### 3.1 Antibody and TCR Homology Modeling

Antibody and TCR structural model were built based on its amino acid sequences using MODELLER. All modeled segments were then assembled to construct the α chimeric chain and β chimeric chain structural model. The relative orientation between the two modeled chains was predicted by the taking the angle of the TCR structure that had the most similar overall sequence. All molecular visualization and analysis work was conducted using PyMOL software (Schrodinger).

### 3.2 DNA Manipulation

The CTCR1 and CTCR2 genes were synthesized by Genewiz Inc. The CH1 and Cλ/Cκ genes were amplified by PCR from existing in-house DNA templates. For those fused with Fc domain chimerics (IgG like ETCR), gene products of light chain shuffles were inserted into linearized vector containing a CMV promoter, a kappa signal peptide and a WPRE regulator while the gene products of heavy chain shuffles were inserted into a linearized vector containing human correspond constant region CH2-CH3, a CMV promoter and a human antibody heavy chain signal peptide. For those not fused with Fc domain chimerics (Fab like ETCR), each of light chain shuffles and heavy chain shuffles were inserted into linearized vector containing a CMV promoter, a kappa signal peptide and a WPRE regulator, respectively. Plasmid ligations, transformations, DNA preparations were performed using standard molecular biology protocols.

### 3.2 Protein Expression

The constructed vectors of heavy chain and light chain were co-transfected into Expi293 cells (Thermofisher Scientific). The ratio of different vectors for co-transfection was optimized according to the expected structure of the ETCR and the initial expression result shown on SDS-PAGE and western blot. The transfection procedure followed the manual provided by the vender. Briefly, 2.5 μg of each plasmid and 13.6 Expifectamine were used to transfect 5 ml volume of 2.94*10⁶ cells. Enhancer 1 and Enhancer 2 were added 20 hours after transfection. The transfected cell were cultured at 37° C., with 8% CO₂, 85% humidity on an orbital shaker, rotating at either 120 rpm (flasks) or 200 rpm (50 ml tubes). Five days after transfection, the supernatants were harvested by centrifuge and cell fragments were removed by 0.22 μm filtering. Before for further testing, the treated supernatants will be concentrated if necessary.

### 3.4 Measurement of ETCR Concentration by ELISA

For IgG like ETCR, ELISA plates were coated with 1 μg/ml anti-human FC antibody in coating buffer (200 mM Na₂CO₃/NaHCO₃, pH 9.2). After incubation over night at 4° C., the plates were washed once with PBST washing buffer using a deep well washer machine (Biotek ELx405). Then the plates were blocked with 1% casein and incubated at room temperature for 1 hour. The plates were washed 3 times with washing buffer, 100 μl positive control (if there is), negative control (if there is) and the diluted samples were added, incubated at room temperature for 1 hour. The plates were washed 3 times with washing buffer, and 100 μl the HRP conjugated anti-lambda antibody or anti kappa antibody were added, incubated at room temperature for 1 hour. The plates were washed 6 times with washing buffer, 100 μl TMB was added and incubated for 10 min, then 100 μl stop solution (2 M HCl, 100 μl/well) was added and the absorbance were read at 450 nm using a plate reader (Molecular Device SpectraMax MV5e).

For Fab like ETCR, ELISA plates were coated with 0.5 Ag/ml anti-His antibody in coating buffer (200 mM Na₂CO₃/NaHCO₃, pH 9.2). After incubation over night at 4° C., the plates were washed once with PBST washing buffer using a deep well washer machine (Biotek ELx405). Then the plates were blocked with 1% casein and incubated at room temperature for 1 hour. The plates were washed 3 times with washing buffer, 100 μl positive control (if there is), negative control (if there is) and the diluted samples were added, incubated at room temperature for 1 hour. The plates were washed 3 times with washing buffer, and 100 μl the HRP conjugated anti-lambda antibody or anti kappa antibody were added, incubated at room temperature for 1 hour. The plates were washed 6 times with washing buffer, 100 μl TMB was added and incubated for 10 min, then 100 μl stop solution (2 M HCl, 100 μl/well) was added and the absorbance were read at 450 nm using a plate reader (Molecular Device SpectraMax MV5e).

### 3.5 Measurement of Target Binding by ELISA

ELISA plates were coated with 2 μg/ml streptavidin (SA) in coating buffer (200 mM Na₂CO₃/NaHCO₃, pH 9.2). After incubation over night at 4° C., the plates were washed once with PBST washing buffer using a deep well washer machine (Biotek ELx405). Then the plates were blocked with 1% casein and incubated at room temperature for 1 hour. The plates were washed 3 times with washing buffer, 2.5 μg/ml antigen HLA*A*02:01 NY-ESO-1 (SLLMWITQC)

HLA*A*02:01 GP100 (YLEPGPVTV) (pHLA, provided by Kactus bio) were added and incubated at room temperature for 1 hour. The plates were washed 3 times with washing buffer, positive control (if there is), negative control (if there is) and the diluted samples were added, incubated at room temperature for 1 hour. The plates were washed 3 times with washing buffer, and 100 μl the HRP conjugated anti-cmyc antibody were added, incubated at room temperature for 1 hour. The plates were washed 6 times with washing buffer, 100 μl TMB was added and incubated for 10 min, then 100 μl stop solution (2 M HCl, 100 μl/well) was added and the absorbance were read at 450 nm using a plate reader (Molecular Device SpectraMax MV5e).

### 4. Results

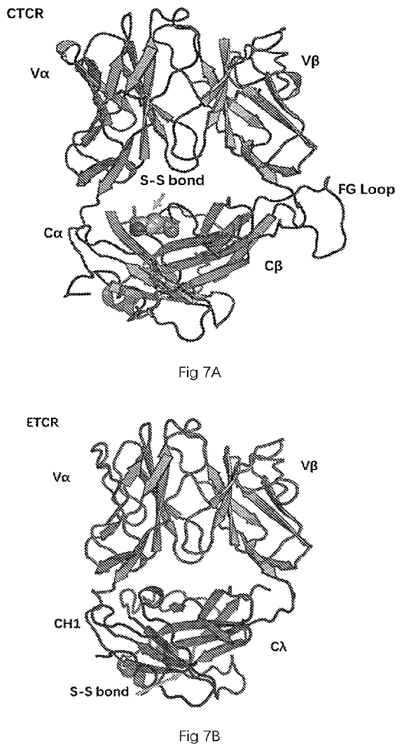

Because TCRs are naturally membrane proteins, transitioning them to a soluble format does not always result in favorable drug-like properties. However, as reported decades ago, by introducing artificial disulfide bond Cα S48-Cβ T57 into non-covalent native TCR Cα-Cβ resulted a stability enhanced soluble TCR format (FIG. 7A, U.S. Pat. No. 7,666,604B2). In contrast to native TCR, native disulfide bond was existed in antibody constant domain (FIG. 7B), may contribute to the stability of the chimerics.

We first evaluated the ability of CH1 and Ca/Cκ to replace the Cα and Cβ. Either CTCR1 and CTCR2 variable domain was shuffled with constant domain from IgG antibody and fused to Fc domain, generating IgG like ETCRs. The expression and binding of these ETCRs were further determined by ELISA. Table 1 &Table 2 listed the construction, expression and binding results of IgG like ETCRs, the harvested supernatant was concentrated 10-times for ELISA analysis. In general, most of the constructions were successfully expressed, however, with relatively low binding signals, indicating that the ETCR part of the chimeric IgG like ETCR may not correctly folded or assembled. Apparently, Fc domain, which expected to further enhance the performance of chimeric TCR, is not capable of stabilizing the ETCR part. Nevertheless, by analyzing the result, we found that “reverse” fusion pattern is better than “normal” fusion pattern: for almost all the tested samples, Vα fused with CH1 and Vβ fused with CL resulted better binding performance than otherwise.

TABLE 1
Construction, expression and binding results of IgG like ETCR using variable domain from CTCR1

| Sample | Interpretation | Expression | Binding |
|---|---|---|---|
| CTCR1 | CTCR1 | / | 3.76 |
| ETCR1 kG1 | CTCR1 constant domain subsituted with Ck and CH1 of IgG1, Vα to CL, Vβ to CH1 | 3.4933 | 0.2096 |
| ETCR1 kG1-Reverse | CTCR1 constant domain subsituted with Ck and CH1 of IgG1, Vα to CH1, Vβ to CL | 3.2181 | 0.3269 |
| ETCR1 kG2 | CTCR1 constant domain subsituted with Ck and CH1 of IgG2, Vα to CL, Vβ to CH1 | 2.774 | 0.0734 |
| ETCR1 kG2-Reverse | CTCR1 constant domain subsituted with Ck and CH1 of IgG2, Vα to CH1, Vβ to CL | 2.1969 | 0.1276 |
| ETCR1 kG4 | CTCR1 constant domain subsituted with Ck and CH1 of IgG4, Vα to CL, Vβ to CH1 | 3.6899 | 0.0971 |
| ETCR1 kG4-Reverse | CTCR1 constant domain subsituted with Ck and CH1 of IgG4, Vα to CH1, Vβ to CL | 3.7256 | 0.7302 |
| ETCR1 λG1 | CTCR1 constant domain subsituted with Cλ and CH1 of IgG1, Vα to CL, Vβ to CH1 | 1.2558 | 0.0503 |
| ETCR1 λG1-Reverse | CTCR1 constant domain subsituted with Cλ and CH1 of IgG1, Vα to CH1, Vβ to CL | 3.4075 | 1.2849 |
| ETCR1 λG2 | CTCR1 constant domain subsituted with Cλ and CH1 of IgG2, Vα to CL, Vβ to CH1 | 0.2434 | 0.0637 |
| ETCR1 λG2-Reverse | CTCR1 constant domain subsituted with Cλ and CH1 of IgG2, Vα to CH1, Vβ to CL | 1.5253 | 0.2113 |
| ETCR1 λG4 | CTCR1 constant domain subsituted with Cλ and CH1 of IgG4, Vα to CL, Vβ to CH1 | 3.3817 | 0.053 |
| ETCR1 λG4-Reverse | CTCR1 constant domain subsituted with Cλ and CH1 of IgG4, Vα to CH1, Vβ to CL | 3.3246 | 0.3695 |

TABLE 2
Construction, expression and binding results of IgG like ETCR using variable domain from CTCR2

| Sample | Interpretation | Expression | Binding |
|---|---|---|---|
| CTCR2 | CTCR2 | / | 3.91 |
| ETCR2 kG1 | CTCR2 constant domain subsituted with Ck and CH1 of IgG1, Vα to CL, Vβ to CH1 | 3.2183 | 0.1514 |
| ETCR2 kG1-Reverse | CTCR2 constant domain subsituted with Ck and CH1 of IgG1, Vα to CH1, Vβ to CL | 3.332 | 0.7986 |
| ETCR2 kG2 | CTCR2 constant domain subsituted with Ck and CH1 of IgG2, Vα to CL, Vβ to CH1 | 3.0539 | 0.0919 |
| ETCR2 kG2-Reverse | CTCR2 constant domain subsituted with Ck and CH1 of IgG2, Vα to CH1, Vβ to CL | 1.7311 | 0.1838 |
| ETCR2 kG4 | CTCR2 constant domain subsituted with Ck and CH1 of IgG4, Vα to CL, Vβ to CH1 | 3.3526 | 0.0904 |
| ETCR2 kG4-Reverse | CTCR2 constant domain subsituted with Ck and CH1 of IgG4, Vα to CH1, Vβ to CL | 3.2736 | 0.2409 |
| ETCR2 λG1 | CTCR2 constant domain subsituted with Cλ and CH1 of IgG1, Vα to CL, Vβ to CH1 | 3.3318 | 0.5929 |
| ETCR2 λG1-Reverse | CTCR2 constant domain subsituted with Cλ and CH1 of IgG1, Vα to CH1, Vβ to CL | 3.4465 | 3.0986 |
| ETCR2 λG2 | CTCR2 constant domain subsituted with Cλ and CH1 of IgG2, Vα to CL, Vβ to CH1 | 1.843 | 0.1581 |
| ETCR2 λG2-Reverse | CTCR2 constant domain subsituted with Cλ and CH1 of IgG2, Vα to CH1, Vβ to CL | 3.1764 | 3.5492 |
| ETCR2 λG4 | CTCR2 constant domain subsituted with Cλ and CH1 of IgG4, Vα to CL, Vβ to CH1 | 2.8399 | 0.2341 |
| ETCR2 λG4-Reverse | CTCR2 constant domain subsituted with Cλ and CH1 of IgG4, Vα to CH1, Vβ to CL | 3.5555 | 2.891 |

Further, more constant domain from IgA, IgD, IgE and IgM were also shuffled with CTCR1 and CTCR2 variable domain, generating Fab-like ETCR (Based on previous result, Fc domain will not be fused to ETCR in further screening and engineering) for extensive screening. The expression and binding of these ETCRs were further determined by ELISA. Table 3 &Table 4 listed the construction, expression and binding results of Fab like ETCR, the harvested supernatant was concentrated 10-times for ELISA analysis. In general, lower expression level as well as binding performance was observed when TCR variable domain was fused with constant domain from IgA, IgD, IgE and IgM.

Based on these result, further engineering will focus on Fab-like ETCR with constant domain comprises IgG CH1 and Cλ/CK, fused in “reverse” pattern.

TABLE 3
Construction, expression and binding results of Fab like ETCR using variable domain from CTCR1

| Sample | Interpretation | Expression | Binding |
|---|---|---|---|
| ETCR1 kA1 | ETCR1 with Ck and CH1 of IgA1, Vα to CL, Vβ to CH1 | 0.1815 | 0.2143 |
| ETCR1 kA1-Reverse | ETCR1 with Ck and CH1 of IgA1, Vα to CH1, Vβ to CL | 1.2005 | 0.3015 |
| ETCR1 kA2 | ETCR1 with Ck and CH1 of IgA2, Vα to CL, Vβ to CH1 | 0.1248 | 0.0754 |
| ETCR1 kA2-Reverse | ETCR1 with Ck and CH1 of IgA2, Vα to CH1, Vβ to CL | 0.3093 | 0.0758 |
| ETCR1 kD | ETCR1 with Ck and CH1 of IgD, Vα to CL, Vβ to CH1 | 0.174 | 0.0718 |
| ETCR1 kD-Reverse | ETCR1 with Ck and CH1 of IgD, Vα to CH1, Vβ to CL | 0.7529 | 0.0885 |
| ETCR1 kE | ETCR1 with Ck and CH1 of IgE, Vα to CL, Vβ to CH1 | 0.1133 | 0.0902 |
| ETCR1 kE-Reverse | ETCR1 with Ck and CH1 of IgE, Vα to CH1, Vβ to CL | 0.1627 | 0.0708 |
| ETCR1 kM | ETCR1 with Ck and CH1 of IgM, Vα to CL, Vβ to CH1 | 0.4116 | 0.2332 |
| ETCR1 kM-Reverse | ETCR1 with Ck and CH1 of IgM, Vα to CH1, Vβ to CL | 2.1272 | 0.1002 |
| ETCR1 λA1 | ETCR1 with Cλ and CH1 of IgA1, Vα to CL, Vβ to CH1 | 0.0916 | 0.1084 |
| ETCR1 λA1-Reverse | ETCR1 with Cλ and CH1 of IgA1, Vα to CH1, Vβ to CL | 0.1936 | 0.415 |
| ETCR1 λA2 | ETCR1 with Cλ and CH1 of IgA2, Vα to CL, Vβ to CH1 | 0.1244 | 0.0729 |
| ETCR1 λA2-Reverse | ETCR1 with Cλ and CH1 of IgA2, Vα to CH1, Vβ to CL | 0.1195 | 0.0744 |
| ETCR1 λD | ETCR1 with Cλ and CH1 of IgD, Vα to CL, Vβ to CH1 | 0.1021 | 0.0712 |
| ETCR1 λD-Reverse | ETCR1 with Cλ and CH1 of IgD, Vα to CH1, Vβ to CL | 0.1533 | 0.0884 |
| ETCR1 λM | ETCR1 with Cλ and CH1 of IgE, Vα to CL, Vβ to CH1 | 0.0787 | 0.0824 |
| ETCR1 λM-Reverse | ETCR1 with Cλ and CH1 of IgE, Vα to CH1, Vβ to CL | 0.0884 | 0.0965 |
| ETCR1 λE | ETCR1 with Cλ and CH1 of IgM, Vα to CL, Vβ to CH1 | 0.1341 | 0.1182 |
| ETCR1 λE-Reverse | ETCR1 with Cλ and CH1 of IgM, Vα to CH1, Vβ to CL | 0.1984 | 0.0931 |

TABLE 4
Construction, expression and binding results of Fab like ETCR using variable domain from CTCR2

| Sample | Interpretation | Expression | Binding |
|---|---|---|---|
| ETCR2 kA1 | ETCR2 with Ck and CH1 of IgA1, Vα to CL, Vβ to CH1 | 0.2157 | 0.1387 |
| ETCR2 kA1-Reverse | ETCR2 with Ck and CH1 of IgA1, Vα to CH1, Vβ to CL | 0.6928 | 0.1367 |
| ETCR2 kA2 | ETCR2 with Ck and CH1 of IgA2, Vα to CL, Vβ to CH1 | 0.7006 | 0.149 |
| ETCR2 kA2-Reverse | ETCR2 with Ck and CH1 of IgA2, Vα to CH1, Vβ to CL | 0.8679 | 0.119 |
| ETCR2 kD | ETCR2 with Ck and CH1 of IgD, Vα to CL, Vβ to CH1 | 0.5681 | 0.1236 |
| ETCR2 kD-Reverse | ETCR2 with Ck and CH1 of IgD, Vα to CH1, Vβ to CL | 0.215 | 0.1159 |
| ETCR2 kE | ETCR2 with Ck and CH1 of IgE, Vα to CL, Vβ to CH1 | 0.1684 | 0.264 |
| ETCR2 kE-Reverse | ETCR2 with Ck and CH1 of IgE, Vα to CH1, Vβ to CL | 0.1116 | 0.1336 |
| ETCR2 kM | ETCR2 with Ck and CH1 of IgM, Vα to CL, Vβ to CH1 | 2.5761 | 0.7248 |
| ETCR2 kM-Reverse | ETCR2 with Ck and CH1 of IgM, Vα to CH1, Vβ to CL | 1.4076 | 0.2291 |
| ETCR2 λA1 | ETCR2 with Cλ and CH1 of IgA1, Vα to CL, Vβ to CH1 | 0.1128 | 1.4659 |
| ETCR2 λA1-Reverse | ETCR2 with Cλ and CH1 of IgA1, Vα to CH1, Vβ to CL | 0.1465 | 3.657 |
| ETCR2 λA2 | ETCR2 with Cλ and CH1 of IgA2, Vα to CL, Vβ to CH1 | 0.1427 | 0.1133 |
| ETCR2 λA2-Reverse | ETCR2 with Cλ and CH1 of IgA2, Vα to CH1, Vβ to CL | 0.186 | 0.1194 |
| ETCR2 λD | ETCR2 with Cλ and CH1 of IgD, Vα to CL, Vβ to CH1 | 0.1151 | 0.1038 |
| ETCR2 λD-Reverse | ETCR2 with Cλ and CH1 of IgD, Vα to CH1, Vβ to CL | 0.1094 | 0.1088 |
| ETCR2 λE | ETCR2 with Cλ and CH1 of IgE, Vα to CL, Vβ to CH1 | 0.0982 | 0.1244 |
| ETCR2 λE-Reverse | ETCR2 with Cλ and CH1 of IgE, Vα to CH1, Vβ to CL | 0.099 | 0.2653 |
| ETCR2 λM | ETCR2 with Cλ and CH1 of IgM, Vα to CL, Vβ to CH1 | 0.8161 | 0.3656 |
| ETCR2 λM-Reverse | ETCR2 with Cλ and CH1 of IgM, Vα to CH1, Vβ to CL | 0.3594 | 0.1669 |

### Example 3: Design and Engineering of Conjunction Domain of ETCR

Generally, conjunction domain between variable domain and constant domain in both TCR and antibody are important for their stabilization and function. However, the linker of IgGs and TCRs are somewhat different. Therefore, linkers with different types and lengths were inserted between the variable domain and constant domain of each chain. Various ETCRs were generated and tested for their expression level and binding performance.

### Results

Firstly, conventional flexible linkers comprising serine and alanine with different lengths were inserted between the TCR variable domain and the antibody constant domain as linkers (SEQ ID Nos:19-24 for TCR Vα-CH1 infusion, and SEQ ID Nos:25-30 for TCR Vβ-Cλ/Cκ infusion).

Table 5 listed the exemplary construction, expression and binding results of Fab like ETCR inserted flexible linker between variable and constant domain, the harvested supernatant was concentrated 10-times for ELISA analysis. Conjunction domain comprises SSAS as linkers (SEQ ID No:19 for a chain and SEQ ID No:25 for B chain) shows best expression level and binding signal in all tested chimeric assembles (Table 5), indicated that the steric hindrance of variable domain and the constant domain were not eliminated and the original TCR function was not fully restored although a little flexibility was introduced between domains.

SEQ ID No: 19, Conjunction domain 1 AA:
SSAS
SEQ ID No: 20, Conjunction domain 1 DNA:
tcgtcggcttca
SEQ ID No: 21, Conjunction domain 2 AA:
SSASS
SEQ ID No: 22, Conjunction domain 2 DNA:
tcgtcggcttcatcg
SEQ ID No: 23, Conjunction domain 3 AA:
SSASSS
SEQ ID No: 24, Conjunction domain 3 DNA:
tcgtcggcttcatcgtca
SEQ ID No: 25, Conjunction domain 4 AA:
SSASKAA
SEQ ID No: 26, Conjunction domain 4 DNA:
agttcggcctcaaaggctgcc
SEQ ID No: 27, Conjunction domain 5 AA:
SSASSKAA
SEQ ID No: 28, Conjunction domain 5 DNA:
tcgtcggcttcatcgaaggctgcc
SEQ ID No: 29, Conjunction domain 6 AA:
SSASSSKAA
SEQ ID No: 30, Conjunction domain 6 DNA:
tcgtcggcttcatcgtcaaaggctgcc

TABLE 5
Construction, expression and binding results of Fab like ETCR
inserted flexible linker between variable and constant domain

| | Expression | Binding | | Expression | Binding |
|---|---|---|---|---|---|
| Sample | (nM) | (OD450) | Sample | (nM) | (OD450) |
| ETCR1-λG1-Reverse | 0.8173 | 0.2309 | ETCR2-λG1-Reverse | 5.4073 | 0.0708 |
| ETCR1-λG1-Reverse-SSAS | 12.6855 | 2.4810 | ETCR2-λG1-Reverse-SSAS | 0.6955 | 0.6319 |
| ETCR1-λG1-Reverse-SSASS | 1.5553 | 2.6022 | ETCR2-λG1-Reverse-SSASS | 0.9145 | 0.1206 |
| ETCR1-λG1-Reverse-SSASSS | 8.1500 | 2.3259 | ETCR2-λG1-Reverse-SSASSS | 0.9418 | 0.1453 |
| ETCR1-λG4-Reverse | 18.0682 | 1.1836 | ETCR2-λG4-Reverse | 4.2455 | 0.2789 |
| ETCR1-λG4-Reverse-SSAS | 248.1482 | 2.4976 | ETCR2-λG4-Reverse-SSAS | 5.6291 | 1.3082 |
| ETCR1-λG4-Reverse-SSASS | 147.8527 | 2.1891 | ETCR2-λG4-Reverse-SSASS | 3.9818 | 0.8260 |
| ETCR1-λG4-Reverse-SSASSS | 103.3091 | 2.2085 | ETCR2-λG4-Reverse-SSASSS | 7.1309 | 0.1787 |

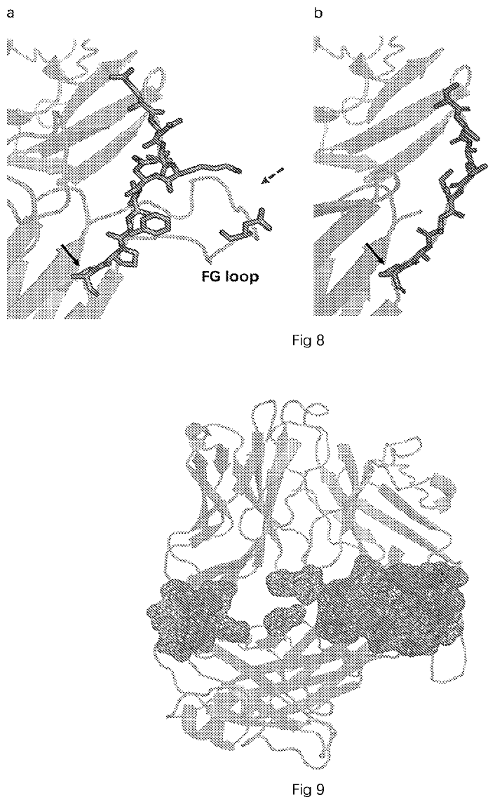

Next, we carefully aligned the sequences of antibody and TCR based on structure alignment, and found that conjunctions defined in germline sequence are not always consistent to domain. we checked how antibody and TCR conjunctions overlapped on the superimposed structures, and estimated the possible replacement using TCR conjunctions to the N terminus of the antibody constant domain (FIG. 8, indicated as black arrow). Particularly, aligning the structures of TCR constant domain with that of antibody revealed that the FG and DE loops of TCR beta chain are significantly longer than the corresponding region in antibody constant domain, and formed strong interactions with the TCR conjunction domain (FIG. 8A, indicated as red arrows). Due to the absence of the long FG and DE loops in the present chimeric ETCRs, key positions in original TCR conjunction domain appeared with unsaturated charged amino acids need to be rationally mutated for enhancing the stability.

Based on such concept, using λG4-Reverse constant domain as exemplary backbone, two conjunction domains (L1 and L2) of β chain (between TCR Vβ domain and Cλ) were firstly designed and test (SEQ ID Nos:33-36, the amino acid sequence of L1 is shown as SEQ ID No:33, amino acid sequence of L2 is shown as SEQ ID No:35, the conjunction domain of chain α is still flexible linker, amino acid sequence of flexible linker is shown as SEQ ID No:19). Table 6 listed the exemplary construction, expression and binding results of Fab like ETCR inserted designed linker between variable and constant domain, the harvested supernatants were concentrated 10 folds for ELISA analysis. Clones of β chain inserted with designed linkers shows better binding signal although expression remained similar compared with A G4-Reverse backbone using both CTCR1 and CTCR1 variable domain, indicated that using designed linkers L1 and L2 in β chain as conjunction domain enabled better structural compatibility and resulted more native-TCR like assembles. Thus, λG4-Reverse with flexible linker and designed linker inserted in chain α and chain β, respectively, were used as exemplary backbone for further engineering.

SEQ ID No: 31, Conjunction domain 7 AA:
EDLNKVFP
SEQ ID No: 32, Conjunction domain 7 DNA:
gaggacctgaacaaggtgttccca
SEQ ID No: 33, Conjunction domain 9 AA:
EDLSNVSP
SEQ ID No: 34, Conjunction domain 9 DNA:
gaggacctgtccaatgtcagtccc
SEQ ID No: 35, Conjunction domain 8 AA:
EDLKNVFP
SEQ ID No: 36, Conjunction domain 8 DNA:
gaggacctgaaaaacgtgttccca

TABLE 6
Construction, expression and binding results of Fab like ETCR
inserted designed linker between variable and constant domain

| | Binding | | Expression | | Binding | | Expression |
|---|---|---|---|---|---|---|---|
| Sample | (OD450) | Sample | (OD450) | Sample | (OD450) | Sample | (OD450) |
| ETCR1-L1 | 3.3159 | ETCR2-L1 | 0.6187 | ETCR2-L1 | 3.8770 | ETCR1-L1 | 0.2357 |
| ETCR1-L2 | 3.1369 | ETCR2-L2 | 0.6624 | ETCR2-L2 | 3.9139 | ETCR1-L2 | 0.2188 |
| ETCR1-λG4- | 2.6968 | ETCR2-λG4- | 0.8880 | ETCR2-λG4- | 3.4848 | ETCR1-λG4- | 0.2344 |
| Reverse | | Reverse | | Reverse | | Reverse | |
| CTCR1 | 4.0000 | CTCR2 | / | CTCR2 | 4.0000 | CTCR1 | / |

### Example 4: Design and Engineering of Vβ-CL Binding Interface of ETCR

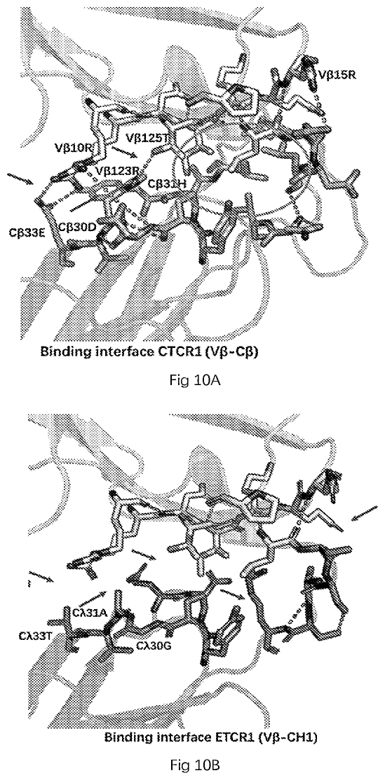

By carefully analyzing the native TCR structures, we found that the binding of variable domain and constant domain of a native TCR is usually contributed by three separated areas, Vα-Ca, Vβ-Cβ and Vα-CR (FIG. 9). Among them, the biggest binding area at Vβ-Cβ was found highly organized, consisting of several H-bonds and salt bridges as well as a hydrophobic core, indicated a very strong binding affinity (FIG. 10A, polar contact is indicated by red arrow and yellow dash line, non-polar contact is indicated by orange circle). Then we superimposed the chimeric ETCR structure to the native TCR, and further noticed that the highly organized interactions of native TCR are totally destroyed by the replacement of Cβ to antibody constant domain Cλ (FIG. 10B). By analyzing the superimposed model, key positions which may contribute to binding between Vβ and Cλ in Cλ were determined and mutagenesis were further designed and tested. The exemplary design and mutations were listed below. IMGT numbering rule was used for all TCR variable domains.

TABLE 7
Exemplary design and mutations

| Design | Mutation |
|---|---|
| bM1 | Ab-Cλ/κ-T33E |
| bM2 | Ab-Cλ/κ-G30D-A31H-T33E |
| bM3 | Ab-Cλ/κ-G30D |
| bM4 | Ab-Cλ/κ-G30D-T33E |
| bM21 | TCR-Vβ-R13K |
| bM22 | TCR-Vβ-R13T |
| bM37 | TCR-Vβ-R90T |
| bM38 | TCR-Vβ-L91I |
| bM39 | TCR-Vβ-M48F |
| bM40 | TCR-Vβ-H54A |
| bM41 | TCR-Vβ-M19Y |
| bM42 | TCR-Vβ-A24K |
| bM43 | TCR-Vβ-A24R |
| bM44 | TCR-Vβ-H54Y |
| bM45 | TCR-Vβ-H54W |
| bM46 | TCR-Vβ-N77E |
| bM47 | TCR-Vβ-R90V |

### Material and Methods

### Protein Purification

6×His-tagged protein was purified by AKTA pure M25 equipped with Ni Sepharose™ Excel chromatography resins (GE Healthcare) in column. Wash buffer A: 50 mM sodium phosphate, 150 mM NaCl, pH 7.2. Wash buffer B: 50 mM sodium phosphate, 150 mM NaCl, 500 mM Imidazole, pH 7.2. The purification process is generally described as following: equilibrate the column at 1 ml/min with wash buffer A. Apply samples at 1 ml/min using sample inlet. Wash the column with wash buffer A at 1 ml/min. Wash the column with 2%, 4%, 10%, 100% wash buffer B. Collect the fractions during the wash process with 1.0 ml/vial.

The pre-purified protein can be further purified by AKTA pure M25 equipped with Superdex™ 75/200 increase chromatography resins (GE Healtcare) in column. Wash buffer: 137 mM sodium phosphate, 2.68 mM NaCl, 1.76 mM KCl, 10 mM KH₂PO₄, 10 mM Na₂HPO₄, pH7.4. The purification process is generally described as following: ultra-filtrate and concentrate the protein to proper loading volume. Wash the column with distilled water. Equilibrate the column with wash buffer. Apply the sample onto the column. Elute the column with the wash buffer until no material appears in the effluent at 0.5 ml/min. The purified protein is stored at −80° C. for future use.

### Quantitation of Purified Protein

A280 was used to preliminary characterization of the purified protein. The absorption value of protein solution at 280 nm was measured by Nanodrop 2000 using 50 mM sodium phosphate, 150 mM NaCl, pH 7.2 as blank buffer. Protein conc. (mg/ml)=A280/Extinction coefficient.

SDS-PAGE was also used for characterization. The running voltage is 200V constant for 35 min and stained using Coomassie blue after electrophoresis.

Purity of the samples was finally determined using size exclusion high performance liquid chromatography. Agilent 1200 HPLC system equipped with TSK GEL G3000SWXL column were used. Wash buffer: 50 mM sodium phosphate. The brief process is generally described as following: equilibrate the column with 50 mM sodium phosphate, 150 mM NaCl, pH7.0. Apply the sample onto the column, UV absorbance at 280 nm was monitored. The purity was estimated by integrating the chromatograms.

### Results

λG4-Reverse with flexible linker SSAS (SEQ ID No:19) and designed linker L1 (SEQ ID No:33) inserted in chain α and chain β, respectively, were used as exemplary backbone for further engineering. Based on the structure analysis, key positions including G30, A31 and T33 in antibody Cλ were identified and mutated to G30D, A31H and T33E respectively, allowing for the generating TCR-like interactions.

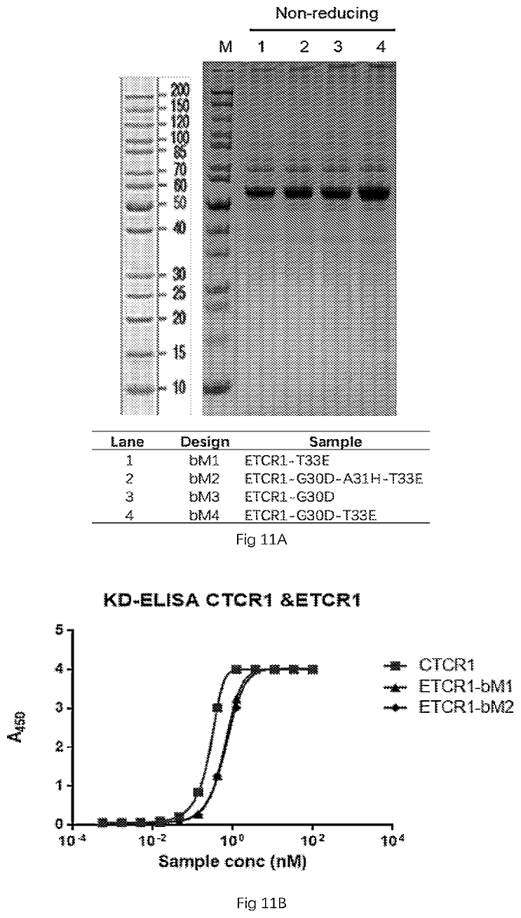

Single mutations as well as combinatorial mutations were generated, expressed, purified and characterized. FIG. 11A shows the electrophoresis result of the supernatant of exemplary mutagenesis, clear bands were visible from the SDS-PAGE, suggested the expression of the chimeric ETCR1 significantly enhanced with these mutations. Further binding ELISA test also revealed a comparable binding performance to native TCR CTCR1 (FIG. 11B, the slightly difference may cause by different detection tag), suggested that the interactions rebuilt between G30D-Vβ123R, A31H-Vβ125T and T33E-Vβ10R via rational mutagenesis strongly stabilized the total ETCR1 structure. Although ETCR1 expressed and functioned well with these mutations, variability was observed in ETCR2 with same mutagenesis.

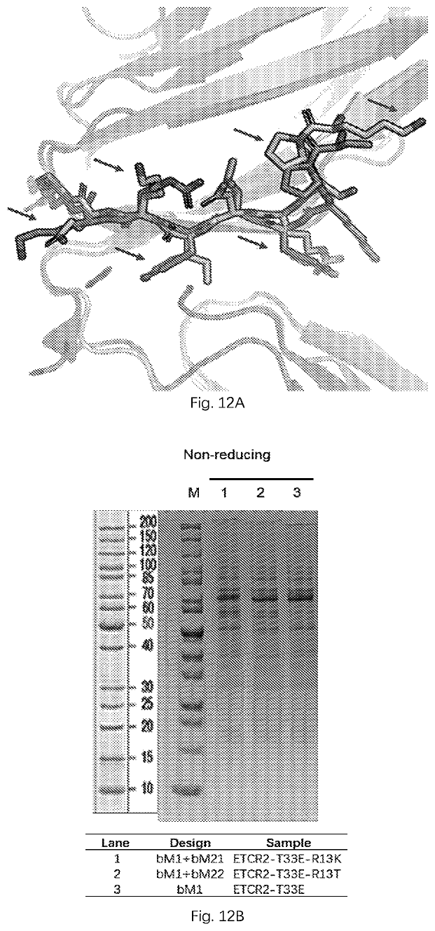

For further increase the compatibility of chain β, binding interface of both ETCR1 and ETCR2 were again superimposed and carefully analyzed. The structure analysis revealed that the framework 1 region (FR1) of TCR variable domain, which is located at the V-C binding interface is significantly different in ETCR1 and ETCR2, providing explanation of aforementioned variabilities (FIG. 12A, the differences are indicated by red arrow). Based on λG4-Reverse with flexible linker SSAS (SEQ ID No:19) and designed linker L1 (SEQ ID No:33) inserted in chain α and chain β format and bM1 design, selected positions according to structural analysis in CTCR2 FR1 were substituted one by one to corresponding amino acid in ETCR 1 FR1 to further increase the compatibility of ETCR2, as a result, one position, R13 of ETCR2 variable β domain (Vβ) was identified. FIG. 12B shows the exemplary electrophoresis result of the supernatant of R13 mutagenesis, clear bands were visible from the SDS-PAGE, suggested the expression of the chimeric ETCR2 significantly enhanced with FR1 mutants compared with bM1. Further Q-ELISA test also revealed an increased expression level of ETCR2-bM1 with R13K/T mutation compared with parental ETCR2-bM1 (957/755 nM Vs 208 nM), indicated that the R13 mutations in VR, together with mutations in Cλ strongly stabilized the total ETCR2 structure.

### Example 5: SPR Analysis of ETCR

The precise binding performance of ETCRs were then determined using SPR technology.

### Method

The binding affinity of ETCR and MHC-peptide (pMHC) antigen was detected using Biacore T200 (or Biocore 8K). The general process is described as following: pMHC antigen was immobilized on CM5 sensor chip (GE). A series concentrations of analyte and running buffer (50 mM sodium phosphate, 150 mM NaCl, 0.05% Tween 20, pH7.4) were injected orderly to chip at a flow rate of 30 μL/min for a 120 second association phase and a 2400 second dissociation phase. After each cycle, the sensor chip surface was regenerated completely with 10 mM glycine (pH 1.5). Surface channels Fc1 without captured ligand was used as control surface for reference subtraction. Final data of each interaction was deducted from reference Fc1 and buffer channel data. Molecular weight of 50.5 kDa was used to calculate the molar concentration of analyte and the experimental data was fitted by Biacore 8K evaluation

### Results

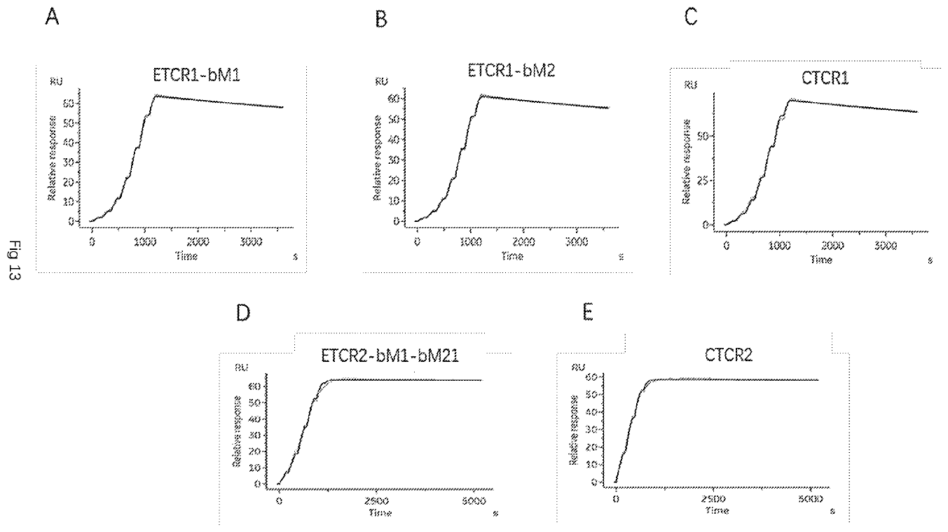

FIG. 13 shows the sensor grams of ETCRs and CTCRs, generally, very similar binding behavior were observed from the sensor grams. Specifically, Table. 13 and Table 8-9 listed the SPR results of the exemplary ETCR1 and ETCR2. The chimeric ETCRs and CTCRs had qualitatively similar binding performance. Particularly, ETCRs shows even better Kₒₙ than CTCRs, which may benefit from more stable, compatible antibody constant domain compared with CTCRs.

TABLE 8
SPR results of exemplary ETCR1 and CTCR1

| Ligand | Analyte | ka (1/Ms) | kd (1/s) | KD (M) |
|---|---|---|---|---|
| HLA*A*02:01- | ETCR1-bM1 | 7.34E+04 | 3.93E−05 | 5.36E−10 |
| NY-ESO-1 | ETCR1-bM2 | 7.13E+04 | 4.13E−05 | 5.79E−10 |
| | CTCR1 | 4.18E+04 | 4.07E−05 | 9.74E−10 |

TABLE 9
SPR results of exemplary ETCR2 and CTCR2

| Ligand | Analyte | ka (1/Ms) | kd (1/s) | KD (M) |
|---|---|---|---|---|
| HLA*A*02:01- | ETCR2- | 2.31E+06 | <1.00E−05* | <4.33E−12 |
| GP100 | bM1-bM21 | | | |
| | CTCR2 | 4.11E+05 | <1.00E−05* | <2.43E−11 |

### Example 6: FACS Analysis of ETCR

The binding performance of ETCRs were then evaluated on tumor cell line using FACS technology.

### Method

The binding ability of designed ETCRs was evaluated using A375 tumor cell line (A375 is a HLA*A*02:01 and NY-ESO-1 dual positive cell line). Cell line were obtained from American Type Culture Collection (ATCC), and were maintained in DMEM medium supplemented with 10% fetal bovine serum (FBS).

Aliquots of 10⁵ cells per well were collected and washed with 1% bovine serum albumin (BSA), followed by the incubation with serial-diluted ETCRs in 96-well round-bottom plate at 4° C. for 1 hour. After being washed triple times with 1% BSA, the plates were further incubated with PE-conjugated goat anti-human c-myc antibody at 4° C. for 30 mins. After the plates were washed triple times again, the cells were analyzed by flow cytometry using a FACSCanto II cytometer (BD Biosciences) and associated fluorescence intensity was quantified using the FlowJo software. Four parameter non-linear regression analysis was used to obtain EC50 values in Prism software (GraphPad Software, Inc).

### Results

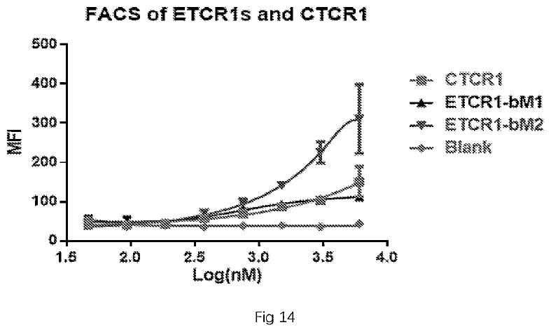

FIG. 14 shows the FACS results of the exemplary ETCR1. The chimeric ETCR1-bM1 and CTCR1 had qualitatively similar binding behaviors. Nevertheless, ETCR1-bM2 had significantly better binding behavior than CTCR1 and ETCR1-bM1, which was not exhibited in ELISA or SPR. We hypothesis that the subtle structural differences of the comprehensive interactions of G30D-Vβ123R, A31H-Vβ125T and T33E-Vβ10R compared to mono interaction T33E-VR 10R can be distinguish under the condition of low antigen density (10-50 copies of antigen per A375 cell).

### Example 7: Design and Engineering of Vβ Framework Domain of ETCR

To further test the compatibility of our chimeric format, we fused our engineered antibody constant domain and introduced mutations in FR1 of TCR variable domain to different TCR germline, however, in terms of one germline pairs, reported as 1G4 previously, failed to produce stable ETCRs. We hypothesis that except the constant domain as well as binding interface, variable domain of TCR may also have impact on the stability of the ETCRs. Thus, the FR regions in Vβ were then comprehensively designed for better stability.

### TCR Sequence

Another HLA*A*02:01 NY-ESO-1 (SLLMWITQC) specific TCR, with a non-native disulfide bond between Cα S48-Cβ T57, named CTCR3 (SEQ ID Nos:45-48), were selected to conduct the study. IMGT numbering rule was used for all TCR variable domains.

Materials and Methods Antibody and TCR Homology Modeling Antibody and TCR structural model were built based on its amino acid sequences using MODELLER. All modeled segments were then assembled to construct the α chimeric chain and β chimeric chain structural model. The relative orientation between the two modeled chains was predicted by the taking the angle of the TCR structure that had the most similar overall sequence. All molecular visualization and analysis work was conducted using PyMOL software (Schrodinger).

### Results

Firstly, variable domain of CTCR3 were amplified and fused to engineered antibody constant domain (λG4-Reverse with flexible linker SSAS (SEQ ID No:19) and designed linker L1 (SEQ ID No:35) inserted in chain α and chain β) comprising bM2 design. However, only roughly 50 nM resulted ETCR-bM2 was expressed and unable to be purified. To investigate if further stabilization of the Vβ domain of TCR could benefit TCR expression, stability and assembly when expressing soluble TCRs in mammalian cells, we used molecular modeling simulations to identify mutations that stabilize the Vβ domain. We scanned over every residue position in the FR region of Vβ domain and used FoldX to model all possible point mutations (except for cysteine) calculated the energies of these mutations. By analyzing and ranking these energies data, 180 mutations from theoretically 1500 mutations were selected for further experimental confirm. Finally, mutation M19Y (bM41), A24K (bM42), A24R (bM43), M48F (bM39), H54Y (bM44), H54W (bM45), H54A (bM40), N77E (bM46), R90T (bM37), R90V (bM47), L911 (bM38) were confirmed that capable of significantly improving the expression level individually. Next, the stabilizing mutations were combined into different variants harboring two to four mutations. Among all the combinations, R90T-L91I (bM37-bM38) resulted the highest expression level, reached 1612 nM. Table 10. listed the SPR results of the exemplary ETCR3. The chimeric ETCRs and CTCRs had qualitatively similar binding performance, indicated that mutations in TCR variable domain also contributes to stabilization of ETCR.

TABLE 10
SPR results of exemplary ETCR3 and CTCR3

| Ligand | Analyte | ka (1/Ms) | kd (1/s) | KD (M) |
|---|---|---|---|---|
| HLA*A*02:01- | ETCR3-bM2-bM37 | 7.04E+05 | 3.75E−03 | 5.33E−09 |
| NY-ESO-1 | ETCR3-bM2-bM37- | 7.02E+05 | 4.12E−03 | 5.87E−09 |
| | bM38 | | | |
| | CTCR3 | 6.13E+05 | 2.65E−03 | 4.31E−09 |

### Example 8: Design and Engineering of Bispecific ETCR

After successfully generated the stably expressed ETCRs, and confirming that the chimeric format was capable of binding the native ligand, we proceeded to construct bispecific formats and tested the in vitro functions. λG4-Reverse with flexible linker SSAS (SEQ ID No:19) and designed linker L1 (SEQ ID No:33) inserted in chain α and chain β, respectively and bM1 design were used as the ETCR1 format.

### Method

### DNA Manipulation and Plasmid Construction

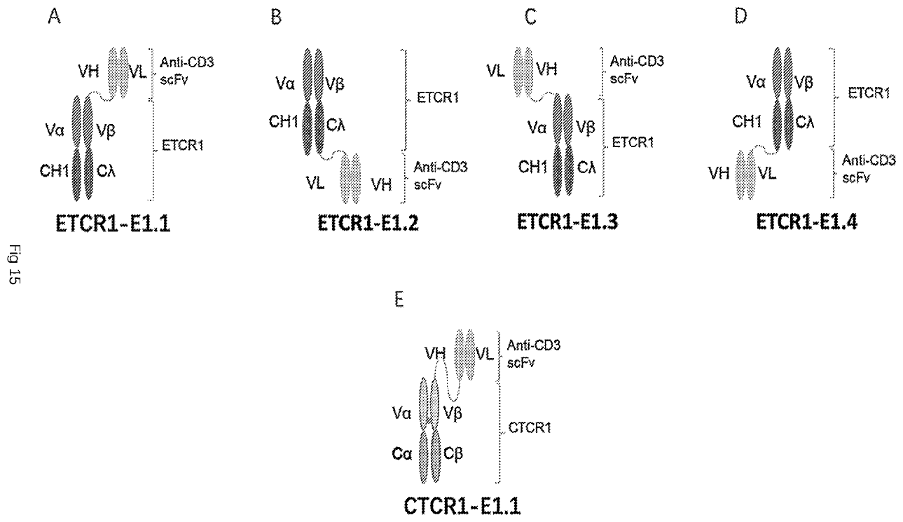

The anti-CD3 scFv antibody (SEQ ID Nos:49-50) genes were synthesized by Genewiz Inc. The gene product of anti-CD3 scFv was amplified and inserted at N terminus of TCR Vβ domain, C terminus of antibody Cλ domain, N terminus of TCR Vα domain, C terminus of antibody CH1 domain, respectively, generating bispecific ETCR1-E1.1, ETCR1-E1.2, ETCR1-E1.3 and ETCR1-E1.4 (FIG. 15A-D). For CTCR, The gene product of anti-CD3 scFv was amplified and inserted at N terminus of TCR Vβ domain, generating CTCR1-E1.1 (FIG. 15E, format as reported). Plasmid ligations, transformations, DNA preparations were performed using standard molecular biology protocols.

Protein expression, purification, and other characterization methods followed aforementioned descriptions.

### Result

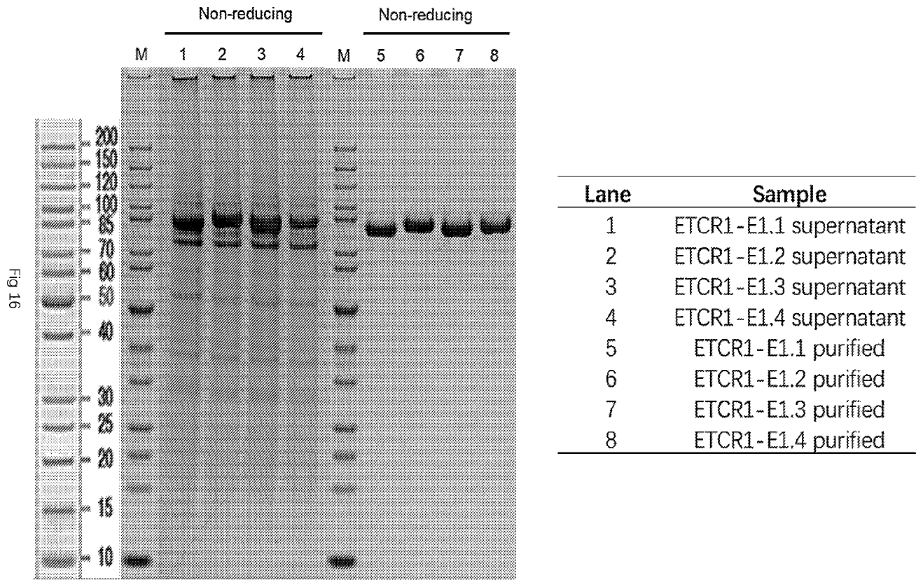

All anti-CD3 scFv antibody (SEQ ID No:50) ETCR fusions were successfully expressed in Expi293 cells and purified. FIG. 16 shows the SDS-PAGE data of the produced bispecific ETCR proteins in supernatant and after purification. The correct molecular weight, i.e. the bands around 78 Kd in non-reduced gel, were all clearly observed. The purified samples were further inspected in SEC-HPLC, which the purity achieved over 99%. The data indicated that the ETCR bispecific proteins were well expressed and assembled.

The binding behavior of all ETCR bispecifics were then test by SPR. Table 11 shows the SPR results of the exemplary ETCR1 bispecifics. The fusion of anti-CD3 scFv at different positions did not significantly affect the binding affinity of ETCR1 are of ETCR1 bispecifics, similar slightly better binding behaviors owing to better Kₒₙ were again observed compared to CTCR1 bispecific. Nevertheless, in terms of the binding behavior of anti-CD3 scFv, variable results were obtained. The binding affinity of ETCR1-E1.1 and ETCR-E1.3 which the anti-CD3 scFv is conjugated at N terminus of TCR Vα and Vβ domain was almost 10 folds higher than ETCR-E1.2 and ETCR-E1.4 which the anti-CD3 scFv is conjugated at C terminus of antibody CH1 and Cλ domain, indicated that the steric hindrance of antibody constant domain seems stronger than the TCR variable domain.

TABLE 11
SPR results of exemplary ETCR1 bispecifics
and CTCR1 bispecific

| Ligand | Analyte | ka (1/Ms) | kd (1/s) | KD (M) |
|---|---|---|---|---|
| CD3 | CTCR1-E1.1 | 1.87E+05 | 2.39E−04 | 1.28E−09 |
| | ETCR1-E1.1 | 2.71E+05 | 2.36E−04 | 8.69E−10 |
| | ETCR1-E1.2 | 2.91E+04 | 2.70E−04 | 9.27E−09 |
| | ETCR1-E1.3 | 2.58E+05 | 2.31E−04 | 8.94E−10 |
| | ETCR1-E1.4 | 1.01E+05 | 2.57E−04 | 2.55E−09 |
| HLA*A*02:01- | CTCR1-E1.1 | 1.59E+04 | 2.36E−05 | 1.48E−09 |
| NY-ESO-1 | ETCR1-E1.1 | 3.12E+04 | 3.08E−05 | 9.87E−10 |
| | ETCR1-E1.2 | 5.84E+04 | 2.76E−05 | 4.73E−10 |
| | ETCR1-E1.3 | 4.79E+04 | 3.98E−05 | 8.32E−10 |
| | ETCR1-E1.4 | 5.32E+04 | 3.09E−05 | 5.81E−10 |

### Example 9: In Vitro T2 Cell Killing Assay of Bispecific ETCR

In vitro function assays were performed to check the activity of the designed ETCR bispecifics in T cell engaged killing of antigen presentation cell T2 loaded with specific peptide. T2 cells are HLA*A*02:01 positive, particularly, deficient in a peptide transporter involved in antigen processing (TAP) and therefore fail to correctly translocate endogenous (processed) peptides to the site of MHC loading in the endoplasmic reticulum Golgi apparatus. Thus, peptide-pulsed T2 cells can be used to monitor the cytotoxic T cells response to an exogenous antigen of interest in a non-competitive environment. Compared with tumor cell line, T2 cells loaded with specific peptide normally provided higher antigen density, and resulted a better killing behavior.

### Method

peripheral blood mononuclear cell (PBMCs) of healthy donors were freshly isolated by Ficoll-Paque PLUS (GE Healthcare-17-1440-03) density centrifugation from heparinized venous blood. After being cultured in RPMI 1640 medium supplemented with 10% FBS, 1% penicillin/Streptomycin solution, 50 units per ml of human IL-2 ligand protein and 10 ng/ml OKT3 antibody for 6 days, the PBMCs were passed through EasySep columns for the enrichment of CD8+ T cells. The CD8+ T cells from the negative selection columns were used as effector cells.

T2 cells (174 9 CEM. T2, ATCC CRL-1992™) was acquired from ATCC and were maintained in IMDM medium supplemented with 20% FBS and penicillin/streptomycin at 37° C., 5% CO₂. Before use, counted and resuspend in medium to 1*10⁶/ml, pulsed with peptides at the peptide concentration of 20 μg/ml for 90 mins in incubator at 37° C., 5% CO₂. Pulsed T2 cells were then labeled with 20 nM Far-Red in DPBS for 30 min and then washed twice and resuspend to designed cell density.

Cells and ETCR bispecifics were then mixed and incubated for designed time. For analysis, 100 μl PI (1:500 diluted in PBS) was add to each well, and run FACS.

### Results

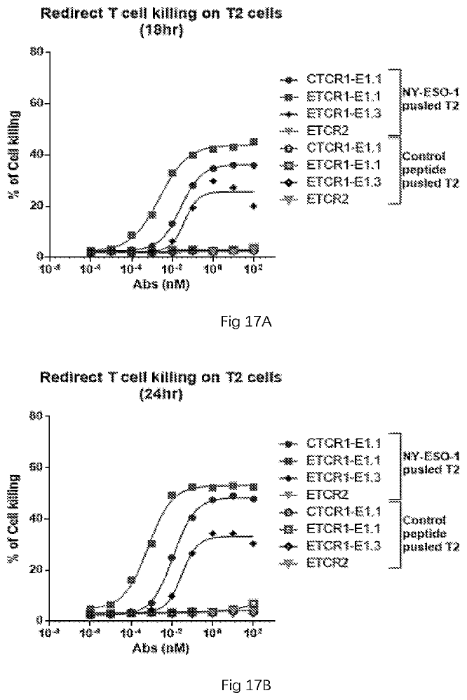

In vitro function assays were performed to check the activity of the designed ETCR bispecifics in T cell engaged killing of antigen presentation cell T2 loaded with specific peptide. T2 cell loaded with irrelevant peptide as well as irrelevant ETCR2 were used as negative control. FIG. 17 shows the dose-dependent cell killing function of exemplary ETCR bispecifics at 18 h and 24 h. No non-specific killing was observed use either negative controls. Besides, ETCR-E1.1 was roughly 20 folds more potent (2.3 pM) than ETCR-E1.3 (39 pM, Table 12), indicated that with anti-CD3 scFv conjugated at N-terminus of TCR Vβ instead of Vα domain resulted the best redirect killing function (no killing effect was observed under same condition using ETCR bispecifics with anti-CD3 scFv conjugated at C terminus of either antibody constant domain, data not show). Particularly, using same anti-CD3 scFv and conjugation position, ETCR-E1.1 (2.3 pM) shows significantly 10 folds more potent killing than CTCR1-E1.1 (25 pM), suggested that the overall stability of chimeric ETCRs is better than CTCRs owing to the engineered antibody constant domain as well as TCR variable domain.

TABLE 12
In vitro T2 cell killing results of exemplary ETCR1 bispecifics and CTCR1 bispecific

| | 18 hr | | | | 24 hr | | | |
|---|---|---|---|---|---|---|---|---|
| | CD8+ T:NY-ESO-1 | | CD8+ T:Control | | CD8+ T:NY-ESO-1 | | CD8+ T:Control | |
| | pulsed T2 = 5:1 | | peptide pulsed T2 = 5:1 | | pulsed T2 = 5:1 | | peptide pulsed T2 = 5:1 | |
| | EC50 | % Max | EC50 | % Max | EC50 | % Max | EC50 | % Max |
| Sample | (nM) | killing | (nM) | killing | (nM) | killing | (nM) | killing |
| CTCR1-E1.1 | 0.025 | 36.1 | NA | 2.6 | 0.012 | 49 | NA | 4.3 |
| ETCR1-E1.1 | 0.0023 | 45.2 | NA | 3.7 | 0.00068 | 53 | NA | 6.9 |
| ETCR1-E1.3 | 0.039 | 29.8 | NA | 2.6 | 0.031 | 34.3 | NA | 3.8 |
| ETCR2 | NA | 2.5 | NA | 2.9 | NA | 4.1 | NA | 4 |

### Example 10: In Vitro Tumor Cell Line Killing Assay of Bispecific ETCR

In vitro function assays were as well performed to check the activity of the designed ETCR bispecifics in T cell engaged killing of tumor cell line A375. A375 tumor cells are HLA*A*02:01 and NY-ESO-1 dual positive with an antigen density between 10-50 copies per cell, suitable for testing the killing of NY-ESO-1 specific TCR bispecifics.

### Method

The method for isolation of CD8+ T cell method is described in Example 9.

A375 cells were acquired from ATCC (ATCC CRL1619™) and maintained in DMEM supplemented with 10% FBS. For killing assay, 50 μl/well diluted ETCR bispecifics were added into black well 96-well flat bottom plate. 50 μl/well isolated CD8+ T cells were added in indicated ratio to 10⁴/well A375 cells and incubated for designed time. For analysis, plates were washed by DPBS once, and the cell variability was detected by Cell Titer-Glo (CTG) assay kit (Promega, Cat. No. G755B).

### Results

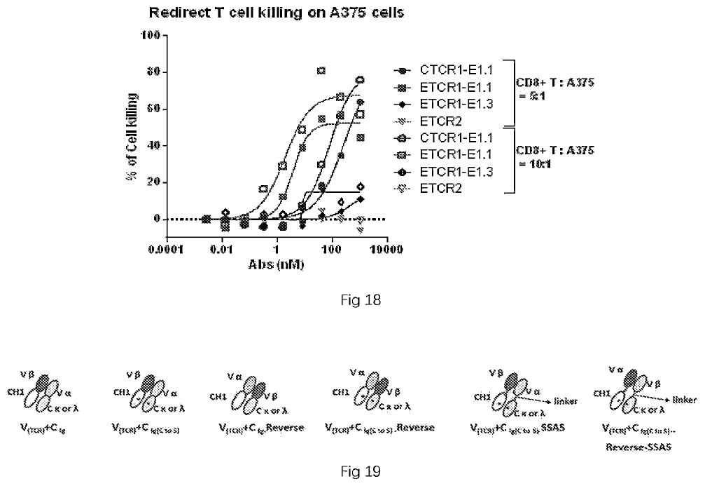

In vitro function assays were performed to check the activity of the designed ETCR bispecifics in T cell engaged killing of tumor cell line A375. Irrelevant ETCR2 were used as negative control. FIG. 18 shows the dose-dependent cell killing function of exemplary ETCRs. No non-specific killing was observed use negative controls. Generally, with a sharply decreased antigen density of A375 tumor cell line compared with T2 cell line, the killing potency of all ETCRs bispecifics were decreased, especially, the killing function of ETCR-E1.3 almost totally lost. Still, ETCR-E1.1 (3.6 nM) showed significantly 70 folds more potent killing than CTCR1-E1.1 (264 nM, Table 13). These data again suggested that the overall stability of chimeric ETCRs is better than CTCRs owing to the engineered antibody constant domain and TCR variable domain, and such advantage is enlarged under low antigen density conditions which frequently appeared in real tumor microenvironment.

TABLE 13
In vitro A375 cell killing results of exemplary
ETCR1 bispecifics and CTCR1 bispecific
72 hr

| | CD8 T cell:A375 = 5:1 | | CD8 T cell:A375 = 10:1 | |
|---|---|---|---|---|
| | EC50 | % Max | EC50 | % Max |
| Sample | (nM) | killing | (nM) | killing |
| CTCR1-E1.1 | 264.6 | 63.9 | 76.31 | 75.8 |
| ETCR1-E1.1 | 3.617 | 56.9 | 1.88 | 80.8 |
| ETCR1-E1.3 | NA | 10.9 | NA | 17.9 |
| ETCR2 | NA | 4.4 | NA | 0.2 |

1. polypeptide complex comprising a first polypeptide comprising, from N-terminus to C-terminus, a first TCR α chain variable domain (TCR Vα) of a first TCR operably linked to a first antibody constant domain (C1), and a second polypeptide comprising, from N-terminus to C-terminus, a first TCR β chain variable domain (TCR Vβ) of a first TCR operably linked to a second antibody constant domain (C2), wherein C1 and C2 are capable of forming a dimer via its native inter-chain bonds and interactions.

2. The polypeptide complex of claim 1, wherein
a) C1 comprises an engineered CH1 domain selecting from the group consisting of IgG1, IgG2, IgG3, IgG4, IgM, IgA1, IgA2, IgD, and IgE; and
C2 comprises an engineered λ or κ light chain constant domain (Cλ domain or Cκ domain) from human immunoglobulin, the Cλ domain is selecting from the group consisting of Cλ1, Cλ2, Cλ3, Cλ6 and Cλ7, the Cκ domain is selecting from the group consisting of Cκ1, Cκ2, Cκ3 and Cκ4; or
b) C1 comprises an engineered λ or κ light chain constant domain (Cλ domain or Cκ domain) from human immunoglobulin, the C domain is selecting from the group consisting of Cλ1, Cλ2, Cλ3, Cλ6 and Cλ7; the Cκ domain is selecting from the group consisting of Cκ1, Cκ2, Cκ3 and Cκ4, and
C2 comprises an engineered CH1 domain selecting from the group consisting of IgG1, IgG2, IgG3, IgG4, IgM, IgA1, IgA2, IgD, and IgE.

3. (canceled)

4. The polypeptide complex of claim 2, wherein the C1 comprises an engineered CH1 comprising the amino acid sequence of any one of SEQ ID Nos:11, 13, 15, and 17, and/or the C2 comprises an engineered Cλ comprising the amino acid sequence of any one of SEQ ID Nos:1, 3, 5, 7 and 9.

5. The polypeptide complex of claim 1, wherein the first TCR Vα is operably linked to C1 through a first conjunction domain, and the first TCR Vβ is operably linked to C2 through a second conjunction domain.

6. The polypeptide complex of claim 5, wherein the C1 comprises an engineered CH1, and the C2 comprises an engineered CX; and wherein the first conjunction domain comprises the amino acid sequence of any one of SEQ ID Nos:19, 21, and 23, and/or the second conjunction domain comprises the amino acid sequence of any one of SEQ ID Nos:25, 27, 29, 31, 33 and 35, optionally, the second conjunction domain comprises EDLXNVXP, wherein X is any amino acid.

7. The polypeptide complex of claim 2, wherein the engineered Cλ comprises mutagenesis at one or more positions selected from 30, 31, 33 of any one of SEQ ID Nos:1, 3, 5, 7, and 9, optionally the engineered Cλ comprises one or more of the following: amino acid D at position 30, amino acid H at position 31 and/or amino acid E at position 33.

8. The polypeptide complex of claim 1, wherein the TCR Vβ comprises mutagenesis at one or more positions selected from 10, 13, 19, 24, 48, 54, 77, 90, 91, 123, and 125 (IMGT numbering) in framework region, optionally, the TCR Vβ comprises at least one mutation at position 13, or comprises at least two mutations at position 90 and 91, optionally the TCR Vβ comprises one or more of the following: amino acid R at position 10, amino acid K at position 13, amino acid T at position 13, amino acid Y at position 19, amino acid K at position 24, amino acid R at position 24, amino acid F at position 48, amino acid Y at position 54, amino acid W at position 54, amino acid A at position 54, amino acid E at position 77, amino acid T at position 90, amino acid V at position 90, amino acid I at position 91, amino acid R at position 123, amino acid T at position 125.

9. A multispecific antigen-binding complex, comprising a first antigen-binding moiety comprising the polypeptide complex of claim 1 and a second antigen-binding moiety, wherein the first antigen-binding moiety has a first antigenic specificity, and the second antigen-binding moiety binds to different epitopes on the first antigen or has a second antigenic specificity which is different from the first antigenic specificity, wherein the second antigen-binding moiety is conjugated at N-terminus or C-terminus of first polypeptide of the first antigen-binding moiety or second polypeptide of the first antigen-binding moiety.

10. (canceled)

11. The multispecific antigen-binding complex of claim 9, wherein one of the first and the second antigenic specificities is directed to a T-cell specific receptor molecule and/or a natural killer cell (NK cell) specific receptor molecule, and the other is directed to a tumor associated antigen and/or tumor neoantigen.

12. The multispecific antigen-binding complex of claim 9, wherein
the first antigen-binding moiety comprises a TCR Vα and a TCR Vβ, the Vα comprises an amino acid sequence selected from SEQ ID Nos:37, 41, and 45, the Vβ comprises an amino acid sequence selected from SEQ ID Nos:39, 43, and 47;
optionally, the second antigen-binding moiety comprises an scFv which comprises the amino acid sequence of SEQ ID No:49.

13. The multispecific antigen-binding complex of claim 9, wherein
the first antigen-binding moiety binds to HLA*A*02:01-NY-ESO-1 peptide (SLLMWITQC) or HLA*A*02:01-GP100 peptide (YLEPGPVTV); and
the second antigen-binding moiety binds to cluster of differentiation 3 (CD3).

14. The multispecific antigen-binding complex of claim 9, wherein the second antigen-binding moiety comprises a single-chain fragment viable (scFv) containing both heavy chain variable domain and a light chain variable domain covalently conjugated via flexible linker.

15. An isolated polynucleotide comprising a nucleotide sequence encoding the polypeptide complex of claim 1.

16. An isolated vector comprising the polynucleotide of claim 15.

17. A host cell comprising the isolated polynucleotide of claim 15.

18. A conjugate comprising the polypeptide complex of claim 1.

19. A method of expressing the polypeptide complex of claim 1, comprising culturing a host cell comprising a polynucleotide(s) encoding the polypeptide complex under the condition at which the polypeptide complex is expressed.

20. A pharmaceutical composition comprising the polypeptide complex of claim 1 or a multispecific antigen-binding complex comprising the polypeptide complex, and a pharmaceutically acceptable carrier.

21. A method of treating a condition including cancer in a subject in need thereof, comprising administrating to the subject a therapeutically effective amount of the multispecific antigen-binding complex of claim 9, optionally the condition can be alleviated, eliminated, treated, or prevented when the first antigen and the second antigen are both modulated.

22. (canceled)

23. A kit comprising the multispecific antigen-binding complex of claim 9.
